(19) United States
(12) Patent Application Publication
(10) Pub. No.: US 2024/0239857 A1
(43) Pub. Date: Jul. 18, 2024

(54) IL-13RALPHA2 TARGETED IMMUNOTOXINS AND METHODS OF USE

(71) Applicant: The United States of America, as represented by the Secretary, Dept. of Health and Human Services, Silver Spring, MD (US)

(72) Inventors: Raj K. Puri, Urbana, MD (US); Bharatkumar H. Joshi, Bethesda, MD (US)

(73) Assignee: The United States of America, as represented by the Secretary, Dept. of Health and Human Services, Silver Spring, MD (US)

(21) Appl. No.: 18/562,128

(22) PCT Filed: May 19, 2022
(86) PCT No.: PCT/US2022/030011

§ 371 Date: Nov. 17, 2023

Related U.S. Application Data
(60) Provisional application No. 63/190,630, filed on May 19, 2021.

(51) Int. Cl. C07K 14/54; A61K 38/00; A61K 47/68; A61P 35/00; C07K 14/47

(52) U.S. Cl. CPC C07K 14/5437; A61K 47/6817; A61K 47/6849; A61P 35/00; C07K 14/4747; A61K 38/00; C07K 2319/55

(57) ABSTRACT

The disclosure relates to immunotoxins, particularly immunotoxins useful for treating IL-13Rα2 expressing cancer, or reducing perineural invasion by an IL-13Rα2 expressing cancer. Immunotoxins that specifically bind IL-13Rα2, nucleic acids encoding the immunotoxins, vectors including the nucleic acids encoding the immunotoxins, and host cells expressing the immunotoxins are provided. Also provided are pharmaceutical compositions including the immunotoxin and methods of treating a subject with cancer, including administering to the subject the immunotoxin alone or in combination with other cancer therapies. Also provided are methods of reducing perineural invasion (PNI) and reducing pain associated with PNI, by administering a therapeutically effective amount of the immunotoxin or IL-13-PE immunotoxin.

### CROSS REFERENCE TO RELATED APPLICATIONS

This claims the benefit of U.S. Provisional Application No. 63/190,630, filed May 19, 2021, which is incorporated by reference herein.

### FIELD OF THE DISCLOSURE

This disclosure relates to immunotherapy, particularly IL-13Rα2 targeting immunotoxins and their use for treating cancer or perineural invasion (PNI).

### BACKGROUND

Pancreatic ductal adenocarcinoma (PDAC) is identified as a highly aggressive cancer, with increased mortality among gastrointestinal cancer patients (Siegel et al. (2019) CA Cancer J Clin, 69: 7-34). While surgical intervention of highly localized pancreatic cancer may lead to a complete cure from this deadly disease, it is commonly seen that pancreatic cancer is characterized by local spread to adjacent tissues or organs with early metastatic lesions in the nearest lymph nodes, liver, surrounding nerves within pancreas, and the peripheral nerve plexus (Bramhall et al. (1998) Hepatobiliary Pancreat Surg, 5:392-401). Most cases of pancreatic cancer present with local invasion of principal arteries and metastasis to the liver at the time of diagnosis. Therefore, only 20% of cases attain the 5-year survival rate of <20% after a complete surgical resection (Feig et al. (2012) Clin Cancer Res, 18:4266-4276; Ozdemir et al., (2014) Cancer Cell, 25:719-734). A potential reason for tumor recurrence may be due to its highly invasive characteristics and attack of surrounding nerves in a process termed perineural invasion (PNI). A high incidence of PNI is observed in PDAC patients, however, PNI is also present in a number of other cancers, including head and neck cancer, prostate cancer, colorectal cancer, breast cancer, stomach cancers and cervical cancers (Badger et al. (2010) Ulster Med J, 79:70-75; Chen et al. (2010) HPB (Oxford), 12:101-108). PNI contributes to the generation of pain and is considered as an indicator of aggressive tumor behavior, and has been shown to correlate with poor prognosis of patients with solid cancers. Thus, there is a need to develop improved treatments for both tumors such as PDAC and for PNI.

### SUMMARY OF THE DISCLOSURE

Disclosed herein are immunotoxins that specifically bind IL-13Rα2. The immunotoxins include a ligand or antibody (or antibody fragment, such as a scFv) that specifically binds IL-13Rα2, linked to a cytotoxic protein (such as a pro-apoptotic protein) of mammalian origin. In some examples, the cytotoxic protein is a human protein, for example, BCL2 associated agonist of cell death (BAD) or Fas-associated death domain (FADD). Also disclosed are nucleic acids and vectors encoding the disclosed immunotoxins. Also disclosed herein are host cells including the disclosed vectors or nucleic acids, and methods of producing the immunotoxin in the host cell. The immunotoxins are useful for treating cancer or tumors, as well as perineural invasion (PNI) and PNI-associated pain caused by IL-13Rα2 positive cancer.

Also disclosed herein are methods of treating an IL-13Rα2-expressing tumor or cancer, methods of reducing perineural invasion of an IL-13Rα2-expressing cancer, and methods of reducing pain resulting from perineural invasion of an IL-13Rα2-expressing cancer in a subject. The methods include administering a therapeutically effective amount of the disclosed immunotoxin, or an IL-13-PE immunotoxin. In some examples, the subject has an IL-13Rα2-expressing cancer or tumor. In some examples, the subject has perineural invasion of an IL-13Rα2-expressing cancer.

The foregoing and other features of the disclosure will become more apparent from the following detailed description, which proceeds with reference to the accompanying figures.

### BRIEF DESCRIPTION OF THE DRAWINGS

FIGS. 1A-1D show that IL-13Rα2 expression in PDAC correlates with pathologic grade and clinical stage of PDAC. FIG. 1A shows hematoxylin and eosin (H&E) staining of PDAC samples with well differentiated, moderately differentiated, and poorly differentiated tumor. FIG. 1B shows immunohistochemical analysis for IL-13Rα2 expression in PDAC and normal pancreas. FIG. 1C shows the extent of immunostaining of IL-13Rα2 in PDAC, as evaluated at 3 levels between 0 and 3+ according to the intensity of staining. FIG. 1D shows the percent positive fields expressing IL-13Rα2 in samples with the indicated pathological grade. Normal pancreas showed negative staining for IL-13Rα2 expression. The samples were viewed at 200× magnification. * P=0.0001.

FIGS. 2A-2D show IL-13Rα2 expressing tumor cells in different clinical stages. FIG. 2A shows hematoxylin and eosin (H&E) staining of PDAC samples with stage I-IV tumors. FIG. 2B shows IHC of IL-13Rα2 expression in PDAC tumors at different stages and normal pancreas. FIG. 2C shows the extent of IHC staining in PDAC, as evaluated at 3 levels between 0 and 3+ according to the intensity of immunostaining. FIG. 2D shows the percent positive fields expressing IL-13Rα2 in samples with different grades. Normal pancreas showed negative staining for IL-13R2e expression. The samples were viewed at 200× magnification. * P=0.0001

FIGS. 3A-3C show Kaplan-Meier survival curves of patients after pancreatic cancer resection. FIG. 3A shows survival of patients with IL-13Rα2-positive tumors and IL-13Rα2− negative tumors. FIG. 3B shows survival of patients with PL− and PL+ tumors (PL=peripancreatic neuroplexus invasion). FIG. 3C shows survival of patients with Ne− and Ne+ tumors (Ne=invasion of nerve in the pancreas).

FIGS. 4A-4D show Kaplan-Meier survival curves of patients after pancreatic cancer resection. FIG. 4A shows survival of patients with PL−/IL-13Rα2− versus PL−/IL-13Rα2+. FIG. 4B shows survival of patients with PL+/IL-13Rα2− versus PL+/IL-13Rα2+. FIG. 4C shows survival of patients with Ne−/IL-13Rα2− versus Ne−/IL-13Rα2+. FIG. 4D shows survival of patients with Ne+/IL-13Rα2− versus Ne+/IL-13Rα2+ PDAC tumors.

FIGS. 5A-5D show in situ hybridization (ISH) analysis to detect IL-13Rα2 mRNA expressing tumors with different pathological grades. FIG. 5A shows H&E staining of PDAC samples with well-, moderately-, or poorly-differentiated grade. FIG. 5B shows ISH of IL-13Rα2 expression in normal and PDAC samples with different pathological grades. FIG. 5C shows the extent of ISH staining in PDAC, as evaluated at 3 levels between 0 and 3+ according to the intensity of immunostaining. FIG. 5D shows the percent positive fields expressing IL-13Rα2 in PDAC samples with different grades. Normal pancreas showed negative staining for IL-13Rα2 mRNA. * P=0.0001.

FIGS. 6A-6D shows that IL-13Rα2 mRNA is detected by ISH in different clinical stages of PDAC. FIG. 6A shows H&E staining of PDAC samples with stage I-IV tumors. FIG. 6B shows IL-13Rα2 mRNA expression by ISH in pancreatic adenocarcinoma and normal pancreas. FIG. 6C shows the extent of hybridization of PDAC, as evaluated at 3 levels between 0 and 3+ according to the intensity of fluorescent anti-sense probe. FIG. 6D shows the percent positive fields expressing IL-13Rα2 in samples with different clinical stages. Normal pancreas showed negative staining for IL-13Rα2 expression. * P=0.0001.

FIG. 7 shows the nucleic acid sequence of an exemplary IL-13-BAD immunotoxin (SEQ ID NO: 2).

FIG. 8 shows the amino acid sequence of an exemplary IL-13-BAD immunotoxin (SEQ ID NO: 1).

FIG. 9 shows the isoelectric point (pl) and Molecular Weight Determination of IL-13-BAD.

FIG. 10 shows a schematic of an exemplary IL-13-BAD construct and cloning into pET24a vector.

FIG. 11 shows cytotoxic activity of IL-13-BAD in IL-13Rα2 positive human glioma cell lines (U251 and A172) after 5 days of culture in the presence of different concentrations of IL-13-BAD. T98G is an IL-13Rα2 negative glioma control. The number of viable cells were counted by trypan blue exclusion on day 5 and shown as % of control.

FIG. 12 shows cytotoxic activity of IL-13-BAD in IL-13Rα2 positive human pancreatic cancer cell line (HS766) after 5 days of culture in the presence of different concentrations of IL-13-BAD. HPAF-II is an IL-13Rα2 negative pancreatic tumor cell line control. The number of viable cells were counted by trypan blue exclusion on day 5 and shown as % of control.

FIG. 13 shows cytotoxic activity of IL-13-BAD in IL-13Rα2 positive squamous cell carcinoma of head and neck (SCCHN) tumor cell lines (HN 12 and YCUM911) after 5 days of culture in the presence of different concentrations of IL-13-BAD. RPMI 2650 is an IL-13Rα2 negative SCCHN control. The number of viable cells were counted by trypan blue exclusion on day 5 and shown as % of control.

FIG. 14 shows that the IL-13-BAD immunotoxin blocks colony forming ability of IL-13Rα2 positive but not IL-13Rα2 negative human tumor cells in vitro.

FIG. 15 shows the nucleic acid sequence of an exemplary IL-13-FADD immunotoxin (SEQ ID NO: 4 is shown).

FIG. 16 shows the amino acid sequence of an exemplary IL-13-FADD immunotoxin (SEQ ID NO: 3).

FIG. 17 shows the isoelectric point (pl) and Molecular Weight Determination of IL-13-FADD.

FIG. 18 shows a schematic of an exemplary IL-13-FADD construct and cloning into pET24a vector.

### SEQUENCE LISTING

Any nucleic acid and amino acid sequences listed herein or in the accompanying Sequence Listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand. The Sequence Listing is submitted as an ASCII text file, “Sequence.txt,” created on May 16, 2022, 32,768 bytes, which is incorporated by reference herein.

SEQ ID NO: 1 is the amino acid sequence of an exemplary
IL-13-BAD immunotoxin.
MSPGPVPPSTALRELIEELVNITQNQKAPLCNGSMVWSINLTAGMYCAALESLINVSGCSAI
EKTQRMLSGFCPHKVSAGQFSSLHVRDTKIEVAQFVKDLLLHLKKLFREGRENGSFQIPEFE
PSEQEDSSSAERGLGPSPAGDGPSGSGKHHRQAPGLLWDASHQQEQPTSSSHHGGAGAVEI
RSRHSSYPAGTEDDEGMGEEPSPFRGRSRSAPPNLWAAQRYGRELRRMSDEFVDSFKKGL
PRPKSAGTATQMRQSSSWTRVFQSWWDRNLGRGSSAPSQ
SEQ ID NO: 2 is an exemplary nucleic acid sequence encoding
IL-13-BAD.
ATGTCCCCAGGCCCTGTGCCTCCCTCTACAGCCCTCAGGGAGCTCATTGAGGAGCTGGT
CAACATCACCCAGAACCAGAAGGCTCCGCTCTGCAATGGCAGCATGGTATGGAGCATC
AACCTGACAGCTGGCATGTACTGTGCAGCCCTGGAATCCCTGATCAACGTGTCAGGCT
GCAGTGCCATCGAGAAGACCCAGAGGATGCTGAGCGGATTCTGCCCGCACAAGGTCTC
AGCTGGGCAGTTTTCCAGCTTGCATGTCCGAGACACCAAAATCGAGGTGGCCCAGTTT
GTAAAGGACCTGCTCTTACATTTAAAGAAACTTTTTCGCGAGGGACGGTTCAACGGAT
CCTTCCAGATCCCAGAGTTTGAGCCGAGTGAGCAGGAAGACTCCAGCTCTGCAGAGAG
GGGCCTGGGCCCCAGCCCCGCAGGGGACGGGCCCTCAGGCTCCGGCAAGCATCATCGC
CAGGCCCCAGGCCTCCTGTGGGACGCCAGTCACCAGCAGGAGCAGCCAACCAGCAGC
AGCCATCATGGAGGCGCTGGGGCTGTGGAGATCCGGAGTCGCCACAGCTCCTACCCCG
CGGGGACGGAGGACGACGAAGGGATGGGGGAGGAGCCCAGCCCCTTTCGGGGCCGCT
CGCGCTCGGCGCCCCCCAACCTCTGGGCAGCACAGCGCTATGGCCGCGAGCTCCGGAG
GATGAGTGACGAGTTTGTGGACTCCTTTAAGAAGGGACTTCCTCGCCCGAAGAGCGCG
GGCACAGCAACGCAGATGCGGCAAAGCTCCAGCTGGACGCGAGTCTTCCAGTCCTGGT
GGGATCGGAACTTGGGCAGGGGAAGCTCCGCCCCCTCCCAGTGA
SEQ ID NO: 3 is the amino acid sequence of an exemplary
IL-13-FADD immunotoxin.
MSPGPVPPSTALRELIEELVNITQNQKAPLCNGSMVWSINLTAGMYCAALESLINVSGCSA
IEKTQRMLSGFCPHKVSAGQFSSLHVRDTKIEVAQFVKDLLLHLKKLFREGRFNGSDPFLV
LLHSVSSSLSSSELTELKFLCLGRVGKRKLERVQSGLDLFSMLLEQNDLEPGHTELLRELL
ASLRRHDLLRRVDDFEAGAAAGAAPGEEDLCAAFNVICDNVGKDWRRLARQLKVSDTK
IDSIEDRYPRNLTERVRESLRIWKNTEKENATVAHLVGALRSCQMNLVADLVQEVQQAR
DLQNRSGAMSPMSWNSDASTSEAS
SEQ ID NO: 4 is an exemplary nucleic acid sequence encoding
IL-13-FADD.
ATGTCCCCAGGCCCTGTGCCTCCCTCTACAGCCCTCAGGGAGCTCATTGAGGAGCTGGT
CAACATCACCCAGAACCAGAAGGCTCCGCTCTGCAATGGCAGCATGGTATGGAGCATC
AACCTGACAGCTGGCATGTACTGTGCAGCCCTGGAATCCCTGATCAACGTGTCAGGCT
GCAGTGCCATCGAGAAGACCCAGAGGATGCTGAGCGGATTCTGCCCGCACAAGGTCTC
AGCTGGGCAGTTTTCCAGCTTGCATGTCCGAGACACCAAAATCGAGGTGGCCCAGTTT
GTAAAGGACCTGCTCTTACATTTAAAGAAACTTTTTCGCGAGGGACGGTTCAACGGAT
CCGACCCGTTTCTGGTGCTGCTGCATAGCGTGTCGTCTTCCCTGTCGTCGTCCGAACTG
ACCGAACTGAAATTCCTGTGTCTGGGCCGTGTGGGTAAACGCAAACTGGAACGTGTTC
AATCGGGCCTGGACCTGTTTAGCATGCTGCTGGAACAGAACGATCTGGAACCGGGTCA
TACCGAACTGCTGCGCGAACTGCTGGCATCTCTGCGTCGCCACGACCTGCTGCGTCGC
GTGGATGACTTTGAAGCTGGTGCAGCAGCAGGTGCTGCACCGGGTGAAGAAGATCTGT
GCGCCGCATTCAACGTTATTTGTGACAATGTCGGCAAAGATTGGCGTCGCCTGGCCCG
TCAGCTGAAAGTCTCTGATACCAAAATTGACAGTATCGAAGATCGTTATCCGCGCAAC
CTGACGGAACGTGTTCGCGAATCGCTGCGCATCTGGAAAAACACCGAAAAAGAAAAT
GCTACGGTCGCGCATCTGGTGGGCGCGCTGCGTAGTTGCCAGATGAATCTGGTTGCTG
ACCTGGTGCAAGAAGTTCAGCAAGCACGTGATCTGCAGAACCGTTCCGGTGCAATGAG
TCCGATGTCCTGGAATTCAGATGCCAGCACGTCTGAAGCATCATAA
SEQ ID NO: 5 is the amino acid sequence of an exemplary
cytotoxic BAD protein.
FQIPEFEPSEQEDSSSAERGLGPSPAGDGPSGSGKHHRQAPGLLWDASHQQEQPTSSSHHG
GAGAVEIRSRHSSYPAGTEDDEGMGEEPSPFRGRSRSAPPNLWAAQRYGRELRRMSDEFV
DSFKKGLPRPKSAGTATQMRQSSSWTRVFQSWWDRNLGRGSSAPSQ
SEQ ID NO: 6 is an exemplary nucleic acid sequence encoding
a cytotoxic BAD protein.
TTCCAGATCCCAGAGTTTGAGCCGAGTGAGCAGGAAGACTCCAGCTCTGCAGAGAGGG
GCCTGGGCCCCAGCCCCGCAGGGGACGGGCCCTCAGGCTCCGGCAAGCATCATCGCCA
GGCCCCAGGCCTCCTGTGGGACGCCAGTCACCAGCAGGAGCAGCCAACCAGCAGCAG
CCATCATGGAGGCGCTGGGGCTGTGGAGATCCGGAGTCGCCACAGCTCCTACCCCGCG
GGGACGGAGGACGACGAAGGGATGGGGGAGGAGCCCAGCCCCTTTCGGGGCCGCTCG
CGCTCGGCGCCCCCCAACCTCTGGGCAGCACAGCGCTATGGCCGCGAGCTCCGGAGGA
TGAGTGACGAGTTTGTGGACTCCTTTAAGAAGGGACTTCCTCGCCCGAAGAGCGCGGG
CACAGCAACGCAGATGCGGCAAAGCTCCAGCTGGACGCGAGTCTTCCAGTCCTGGTGG
GATCGGAACTTGGGCAGGGGAAGCTCCGCCCCCTCCCAGTGA
SEQ ID NO: 7 is the amino acid sequence of an exemplary
cytotoxic FADD protein.
DPFLVLLHSVSSSLSSSELTELKFLCLGRVGKRKLERVQSGLDLFSMLLEQNDLEPGHTELL
RELLASLRRHDLLRRVDDFEAGAAAGAAPGEEDLCAAFNVICDNVGKDWRRLARQLKVS
DTKIDSIEDRYPRNLTERVRESLRIWKNTEKENATVAHLVGALRSCQMNLVADLVQEVQQ
ARDLQNRSGAMSPMSWNSDASTSEAS
SEQ ID NO: 8 is an exemplary nucleic acid sequence encoding a
cytotoxic FADD protein.
GACCCGTTTCTGGTGCTGCTGCATAGCGTGTCGTCTTCCCTGTCGTCGTCCGAACTGAC
CGAACTGAAATTCCTGTGTCTGGGCCGTGTGGGTAAACGCAAACTGGAACGTGTTCAA
TCGGGCCTGGACCTGTTTAGCATGCTGCTGGAACAGAACGATCTGGAACCGGGTCATA
CCGAACTGCTGCGCGAACTGCTGGCATCTCTGCGTCGCCACGACCTGCTGCGTCGCGT
GGATGACTTTGAAGCTGGTGCAGCAGCAGGTGCTGCACCGGGTGAAGAAGATCTGTGC
GCCGCATTCAACGTTATTTGTGACAATGTCGGCAAAGATTGGCGTCGCCTGGCCCGTC
AGCTGAAAGTCTCTGATACCAAAATTGACAGTATCGAAGATCGTTATCCGCGCAACCT
GACGGAACGTGTTCGCGAATCGCTGCGCATCTGGAAAAACACCGAAAAAGAAAATGC
TACGGTCGCGCATCTGGTGGGCGCGCTGCGTAGTTGCCAGATGAATCTGGTTGCTGAC
CTGGTGCAAGAAGTTCAGCAAGCACGTGATCTGCAGAACCGTTCCGGTGCAATGAGTC
CGATGTCCTGGAATTCAGATGCCAGCACGTCTGAAGCATCATAAAAGCTT
SEQ ID NO: 9 is the amino acid sequence of an exemplary
matured human IL-13 protein.
MSPGPVPPSTALRELIEELVNITQNQKAPLCNGSMVWSINLTAGMYCAALESLINVSGCSAI
EKTQRMLSGFCPHKVSAGQFSSLHVRDTKIEVAQFVKDLLLHLKKLFREGREN
SEQ ID NO: 10 is an exemplary nucleic acid sequence encoding
matured human IL-13.
ATGTCCCCAGGCCCTGTGCCTCCCTCTACAGCCCTCAGGGAGCTCATTGAGGAGCTGGT
CAACATCACCCAGAACCAGAAGGCTCCGCTCTGCAATGGCAGCATGGTATGGAGCATC
AACCTGACAGCTGGCATGTACTGTGCAGCCCTGGAATCCCTGATCAACGTGTCAGGCT
GCAGTGCCATCGAGAAGACCCAGAGGATGCTGAGCGGATTCTGCCCGCACAAGGTCTC
AGCTGGGCAGTTTTCCAGCTTGCATGTCCGAGACACCAAAATCGAGGTGGCCCAGTTT
GTAAAGGACCTGCTCTTACATTTAAAGAAACTTTTTCGCGAGGGACGGTTCAAC
SEQ ID NO: 11 is the amino acid sequence of an exemplary
IL-13Ra2 scFv.
QVQLVQSGAEVKKPGASVKVSCKASGYTFTSYAMHWVRQAPGQRLEWMGWINAGNGN
TKYSQKFQGRVTITRDTSASTAYMELSSLRSEDTAVYYCARMNHMIPLKAWGQGTLVTVS
SGGGGSGGGGSGGSALAIQMTQSPSSLSASVGDRVTITCRASQGIRNDLGWYQQKPGKAP
KLLIYAASSLQSGVPSRFSGSGSGTDFTLTISSLQPEDFATYYCLQMYNYRTFGQGTKLEIK
RA
SEQ ID NO: 12 is an exemplary nucleic acid sequence encoding
the IL-13Ra2 scFv of SEQ ID NO: 11.
AGGTGCAGCTGGTGCAGAGCGGAGCAGAGGTGAAGAAGCCAGGAGCCTCTGTGAAGG
TGAGCTGCAAGGCCTCCGGCTACACATTCACCTCCTATGCCATGCACTGGGTGAGACA
GGCACCTGGACAGAGGCTGGAGTGGATGGGCTGGATCAACGCCGGCAACGGCAATAC
AAAGTACTCTCAGAAGTTTCAGGGCCGCGTGACAATCACCCGGGACACATCCGCCTCT
ACCGCCTATATGGAGCTGAGCTCCCTGCGGTCCGAGGATACCGCCGTGTACTATTGCG
CCAGAATGAATCACATGATCCCACTGAAGGCATGGGGACAGGGCACACTGGTGACCG
TGTCTAGCGGAGGAGGAGGCAGCGGAGGAGGAGGCTCCGGCGGCTCTGCCCTGGCCA
TCCAGATGACCCAGTCCCCATCCTCTCTGAGCGCCTCCGTGGGCGACCGCGTGACAAT
CACCTGTCGGGCCAGCCAGGGCATCAGAAACGATCTGGGCTGGTACCAGCAGAAGCC
CGGCAAGGCCCCTAAGCTGCTGATCTATGCAGCAAGCTCCCTGCAGTCTGGAGTGCCT
AGCCGGTTCTCTGGCAGCGGCTCCGGAACAGACTTTACACTGACCATCTCTAGCCTGC
AGCCAGAGGATTTCGCCACCTACTATTGCCTGCAGATGTACAATTATAGAACATTTGG
CCAGGGCACCAAGCTGGAGATCAAGAGGGCC
SEQ ID NO: 13 is a forward primer sequence to amplify human IL-13.
TAATTTGCCCATATGTCCCCAGGCCCT
SEQ ID NO: 14 is a reverse primer sequence to amplify human IL-13.
GAAGTTGGATCCTGTTGAACCGTCCCTCGC
SEQ ID NO: 15 is a forward primer sequence to amplify human FADD.
GTTAAAGGATCCGACCCGTTTCTGCTC
SEQ ID NO: 16 is a reverse primer sequence to amplify human FADD.
TCCTGTAAGCTTTCAGGACGCTTC
SEQ ID NO: 17 is the amino acid sequence of an exemplary
IL-13-PE immunotoxin.
MSPGPVPPSTALRELIEELVNITQNQKAPLCNGSMVWSINLTAGMYCAALESLINVSGCSAI
EKTQRMLSGFCPHKVSAGQFSSLHVRDTKIEVAQFVKDLLLHLKKLFREGRENKASGGPE
GGSLAALTAHQACHLPLETFTRHRQPRGWEQLEQCGYPVQRLVALYLAARLSWNQVDQV
IRNALASPGSGGDLGEAIREQPEQARLALTLAAAESERFVRQGTGNDEAGAANGPADSGD
ALLERNYPTGAEFLGDGGDVSFSTRGTQNWTVERLLQAHRQLEERGYVFVGYHGTFLEA
AQSIVFGGVRARSQDLDAIWRGFYIAGDPALAYGYAQDQEPDARGRIRNGALLRVYVPRS
SLPGFYRTSLTLAAPEAAGEVERLIGHPLPLRLDAITGPEEEGGRLETILGWPLAERTVVIPS
AIPTDPRNVGGDLDPSSIPDQEQAISALPDYASQPGQPPREDLR
SEQ ID NO: 18 is an exemplary nucleic acid sequence encoding
an IL-13-PE immunotoxin.
ATGTCCCCAGGCCCTGTGCCTCCCTCTACAGCCCTCAGGGAGCTCATTGAGGAGCTGGT
CAACATCACCCAGAACCAGAAGGCTCCGCTCTGCAATGGCAGCATGGTATGGAGCATC
AACCTGACAGCTGGCATGTACTGTGCAGCCCTGGAATCCCTGATCAACGTGTCAGGCT
GCAGTGCCATCGAGAAGACCCAGAGGATGCTGAGCGGATTCTGCCCGCACAAGGTCTC
AGCTGGGCAGTTTTCCAGCTTGCATGTCCGAGACACCAAAATCGAGGTGGCCCAGTTT
GTAAAGGACCTGCTCTTACATTTAAAGAAACTTTTTCGCGAGGGACGGTTCAACAAAG
CTTCCGGAGGTCCCGAGGGCGGCAGCCTGGCCGCGCTGACCGCGCACCAGGCTTGCCA
CCTGCCGCTGGAGACTTTCACCCGTCATCGCCAGCCGCGCGGCTGGGAACAACTGGAG
CAGTGCGGCTATCCGGTGCAGCGGCTGGTCGCCCTCTACCTGGCGGCGCGGCTGTCGT
GGAACCAGGTCGACCAGGTGATCCGCAACGCCCTGGCCAGCCCCGGCAGCGGCGGCG
ACCTGGGCGAAGCGATCCGCGAGCAGCCGGAGCAGGCCCGTCTGGCCCTGACCCTGGC
CGCCGCCGAGAGCGAGCGCTTCGTCCGGCAGGGCACCGGCAACGACGAGGCCGGCGC
GGCCAACGGCCCGGCGGACAGCGGCGACGCCCTGCTGGAGCGCAACTATCCCACTGG
CGCGGAGTTCCTCGGCGACGGCGGCGACGTCAGCTTCAGCACCCGCGGCACGCAGAAC
TGGACGGTGGAGCGGCTGCTCCAGGCGCACCGCCAACTGGAGGAGCGCGGCTATGTGT
TCGTCGGCTACCACGGCACCTTCCTCGAAGCGGCGCAAAGCATCGTCTTCGGCGGGGT
GCGCGCGCGCAGCCAGGACCTCGACGCGATCTGGCGCGGTTTCTATATCGCCGGCGAT
CCGGCGCTGGCCTACGGCTACGCCCAGGACCAGGAACCCGACGCACGCGGCCGGATC
CGCAACGGTGCCCTGCTGCGGGTCTATGTGCCGCGCTCGAGCCTGCCGGGCTTCTACC
GCACCAGCCTGACCCTGGCCGCGCCGGAGGCGGCGGGCGAGGTCGAACGGCTGATCG
GCCATCCGCTGCCGCTGCGCCTGGACGCCATCACCGGCCCCGAGGAGGAAGGCGGGC
GCCTGGAGACCATTCTCGGCTGGCCGCTGGCCGAGCGCACCGTGGTGATTCCCTCGGC
GATCCCCACCGACCCGCGCAACGTCGGCGGCGACCTCGACCCGTCCAGCATCCCGGAC
CAAGAACAGGCGATCAGCGCCCTGCCGGACTACGCCAGCCAGCCCGGCCAACCGCCG
CGCGAGGACCTGCGTTAACTGCCGCGACCGGCCGGCTCCCTTCGCAGGAGCCGGCCTT
CTCGGGGCCTGGCCATACATCAGGTTTTCCTGATGCCAGCCCAATCGAATATGAATTC
SEQ ID NO: 19 is the amino acid sequence of an exemplary full
length cytotoxic BAD protein.
MFQIPEFEPSEQEDSSSAERGLGPSPAGDGPSGSGKHHRQAPGLLWDASHQQEQPTSSSHH
GGAGAVEIRSRHSSYPAGTEDDEGMGEEPSPFRGRSRSAPPNLWAAQRYGRELRRMSDEF
VDSFKKGLPRPKSAGTATQMRQSSSWTRVFQSWWDRNLGRGSSAPSQ
SEQ ID NO: 20 is the amino acid sequence of an exemplary full
length cytotoxic FADD protein.
MDPFLVLLHSVSSSLSSSELTELKFLCLGRVGKRKLERVQSGLDLFSMLLEQNDLEPGHTEL
LRELLASLRRHDLLRRVDDFEAGAAAGAAPGEEDLCAAFNVICDNVGKDWRRLARQLKV
SDTKIDSIEDRYPRNLTERVRESLRIWKNTEKENATVAHLVGALRSCQMNLVADLVQEVQ
QARDLQNRSGAMSPMSWNSDASTSEAS
SEQ ID NO: 21 is an exemplary amino acid linker.
Gly Gly Gly Gly Ser Gly Gly Gly Gly Ser Gly Gly Gly Gly Ser
SEQ ID NO: 22 is an exemplary amino acid linker.
Thr Arg His Arg Gln Pro Arg Gly Trp Glu Gln Leu
SEQ ID NO: 23 is an exemplary full length human IL-13
protein (unmatured).
MALLLTTVIALTCLGGFASPGPVPPSTALRELIEELVNITQNQKAPLCNGSMVWSINLTAGM
YCAALESLINVSGCSAIEKTQRMLSGFCPHKVSAGQFSSLHVRDTKIEVAQFVKDLLLHLK
KLFREGREN

### DETAILED DESCRIPTION

IL-13Rα2 is a high affinity receptor to the Th2 derived cytokine IL-13 and has been identified as a cancer testis antigen (Deng et al. (2018) Mol Cancer Res, 16:623-633; Taguchi et al. (2014) Cancer Res, 74:4694-4705). It has been previously shown that IL-13Rα2 is overexpressed in numerous solid human cancers such as malignant glioma, squamous cell carcinoma of head and neck, Kaposi's sarcoma, kidney cancer, adrenocortical cancer, and ovarian carcinoma (Joshi et al. (2006) Vitam Horm, 74:479-504; Suzuki et al. (2015) Cytokine, 75:79-88). In contrast, normal immune cells do not express, or only weakly express, IL-13Rα2.

Here, it is shown that IL-13Rα2 is significantly overexpressed in tumor cells invading peripancreatic neuroplexus and nerve endings, which is correlated with poor survival of patients. As PNI is associated with the generation of pain experienced by PDAC (and other) cancer patients, and is associated with IL-13Rα2 overexpression, therapies targeting IL-13Rα2 are provided herein for inhibiting cancer invasion, metastasis, and PNI, in order to improve the survival of PDAC (and other) patients and alleviating their pain.

### I. Terms

Unless otherwise noted, technical terms are used according to conventional usage. Definitions of common terms in molecular biology may be found in Lewin's Genes X, ed. Krebs et al., Jones and Bartlett Publishers, 2009 (ISBN 0763766321); Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, Proteomics and Informatics, 3^{rd} Edition, Springer, 2008 (ISBN: 1402067534), and other similar references.

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. “Comprising A or B” means including A, or B, or A and B. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting. All references, including patent applications and patents, and sequences associated with the National Center for Biotechnology Information (NCBI), GenBank®, or the European Nucleotide Archive (ebi.ac.uk/ena/browser/home) (as of May 19, 2021) are herein incorporated by reference in their entirety.

In order to facilitate review of the various embodiments of the disclosure, the following explanations of specific terms are provided:

Antibody: A polypeptide ligand comprising at least one variable region that recognizes and binds (such as specifically recognizes and specifically binds) an epitope of an antigen. Mammalian immunoglobulin molecules are composed of a heavy (H) chain and a light (L) chain, each of which has a variable region, termed the variable heavy (V_{H}) region and the variable light (V_{L}) region, respectively. Together, the V_{H} region and the V_{L} region are responsible for binding the antigen recognized by the antibody. There are five main heavy chain classes (or isotypes) of mammalian immunoglobulin, which determine the functional activity of an antibody molecule: IgM, IgD, IgG, IgA and IgE.

Antibody variable regions contain “framework” regions and hypervariable regions, known as “complementarity determining regions” or “CDRs.” The CDRs are primarily responsible for binding to an epitope of an antigen. The framework regions of an antibody serve to position and align the CDRs in three-dimensional space. The amino acid sequence boundaries of a given CDR can be readily determined using any of a number of well-known numbering schemes, including those described by Kabat et al. (Sequences of Proteins of Immunological Interest, U.S. Department of Health and Human Services, 1991; the “Kabat” numbering scheme), Chothia et al. (see Chothia and Lesk, J Mol Biol 196:901-917, 1987; Chothia et al., Nature 342:877, 1989; and Al-Lazikani et al., (JMB 273,927-948, 1997; the “Chothia” numbering scheme), and the ImMunoGeneTics (IMGT) database (see, Lefranc, Nucleic Acids Res 29:207-9, 2001; the “IMGT” numbering scheme). The Kabat and IMGT databases are maintained online.

A single-chain antibody (scFv) is a genetically engineered molecule containing the V_{H} and V_{L} domains of one or more antibody(ies) linked by a suitable polypeptide linker as a genetically fused single chain molecule (see, for example, Bird et al., Science, 242:423-426, 1988; Huston et al., Proc. Natl. Acad. Sci., 85:5879-5883, 1988; Ahmad et al., Clin. Dev. Immunol., 2012, doi: 10.1155/2012/980250; Marbry, IDrugs, 13:543-549, 2010). The intramolecular orientation of the V_{H}-domain and the V_{L}-domain in a scFv, is typically not decisive for scFvs. Thus, scFvs with both possible arrangements (V_{H}-domain-linker domain-V_{L}-domain; V_{L}-domain-linker domain-V_{H}-domain) may be used. In a dsFv the V_{H} and V_{L} have been mutated to introduce a disulfide bond to stabilize the association of the chains. Diabodies also are included, which are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90:6444-6448, 1993; Poljak et al., Structure, 2:1121-1123, 1994).

Antibodies also include genetically engineered forms such as chimeric antibodies (such as humanized murine antibodies) and heteroconjugate antibodies (such as bispecific antibodies). See also, Pierce Catalog and Handbook, 1994-1995 (Pierce Chemical Co., Rockford, IL); Kuby, J., Immunology, 3^{rd} Ed., W.H. Freeman & Co., New York, 1997.

BCL2 associated agonist of cell death (BAD): BAD is a Bcl-2 protein family member and plays a role in cellular apoptosis. Specifically, BAD positively regulates cell apoptosis by forming heterodimers with BCL-XL (B-cell lymphoma-extra large) and BCL-2, and reversing their death repressor activity. BAD is also known as BBC2 or BCL2L8. Sequences for BAD are known, for example, see NCBI Reference Sequence NM_004322.3 and NM_032989.3 and for human BAD mRNA sequences, or NCBI Accession NP_116784.1 or NP_004313.1 for human BAD amino acid sequences, herein incorporated by reference in their entirety.

Cancer: A malignant tumor characterized by abnormal or uncontrolled cell growth. Other features often associated with cancer include metastasis, interference with the normal functioning of neighboring cells, release of cytokines or other secretory products at abnormal levels, suppression or aggravation of inflammatory or immunological response, invasion of surrounding or distant tissues or organs, such as lymph nodes, etc. “Metastatic disease” refers to cancer cells that have left the original tumor site and migrated to other parts of the body, for example via the bloodstream or lymph system. In some examples, the cancer is an IL-13Rα2-expressing cancer.

Chimera: A non-naturally occurring nucleic acid or protein that has a sequence made by an artificial combination of two otherwise separated segments of sequence (e.g., fusion of a ligand or antibody fragment with a cytotoxic protein). This artificial combination can be accomplished by chemical synthesis or by the artificial manipulation of isolated segments of nucleic acids, for example, by genetic engineering techniques.

Cytotoxic: An agent that is toxic to living cells. An example of a cytotoxic protein is an apoptotic protein, which induces cellular apoptosis, or a protein that otherwise induces cell death. In some examples, the cytotoxic protein is of mammalian origin, such as from human, mouse, non-human primate, mouse, rat, rabbit, pig, goat, sheep, dog, cat, horse, or cow. Examples of mammalian cytotoxic proteins, including proapoptotic proteins, include caspases, amyloid-B peptide, B-cell lymphoma/leukemia-2 gene (Bcl-2) proteins, p53, heat shock proteins, or an interacting protein thereof that contributes to inducing apoptosis. In some non-limiting examples, the cytotoxic protein is BCL2 associated agonist of cell death (BAD) or Fas-associated death domain (FADD). Cytotoxic proteins include amino acid sequence variants that retain functionality (e.g., cytotoxicity). For example, the cytotoxic protein has at least 70%, at least 80%, at least 90%, at least 95%, at least 98%, at least 99%, or more sequence identity to a cytotoxic protein of mammalian origin and retains cytotoxic activity.

Effective Amount: An amount of an agent, such as a disclosed immunotoxin or IL-13-PE immunotoxin, that is sufficient to treat, reduce, and/or ameliorate the symptoms and/or underlying cause of a disease or pathological condition, such as cancer or perineural invasion in a subject. In a specific non-limiting example, an effective amount is an amount sufficient to inhibit or reduce tumor growth, perineural invasion, or symptoms of a tumor or perineural invasion (such as pain) in the subject.

Fas-associated death domain (FADD): FADD is a tumor necrosis factor receptor superfamily related death protein. It interacts with various cell surface receptors to mediate apoptotic signals. This protein can be recruited through its death domain by TNFRSF6/Fas-receptor, tumor necrosis factor receptor, TNFRSF25, and TNFSF10/TRAIL-receptor, and thus participates in the death signaling initiated by these receptors. Sequences for FADD are known, for example, see NCBI Gene ID: NM_003824.4 for human FADD mRNA or NCBI Accession NP_003815.1 for human FADD amino acid sequence, herein incorporated by reference in their entirety.

Interleukin-13 (IL-13): A cytokine with a high affinity to the IL-13Rα2 receptor. Sequences for IL-13 are known, for example, see NCBI Gene ID: NM_002188.3, NM_001354991.2, NM_001354992.2, and NM_001354993.2, or European Nucleotide Archive (ENA) sequence X69079.1, for human IL-13 mRNA, or NCBI Accession NP_002179.2, NP_001341920.1, NP_001341921.1, NP_001341921.1 for human IL-13 amino acid sequences, all of which are herein incorporated by reference in their entirety. In some examples, “IL-13” refers to matured IL-13, or a portion of IL-13 that specifically binds to the IL-13Rα2 receptor. In some examples, matured IL-13 does not include a signal sequence.

IL-13-PE Immunotoxin: IL-13-PE is a recombinant immunotoxin consisting of IL-13 and a truncated pseudomonas exotoxin. IL-13-PE has been shown to be highly cytotoxic to tumor cells expressing high levels of IL-13Rα2 both in vitro and in vivo. (see U.S. Pat. No. 6,518,061; Debinski et al., Clin Cancer Res. 1:1253-1258, 1995; Debinski et al., Biol Chem. 270:16775-16780, 1995; and Joshi et al. Clin Cancer Res. 8:1948-1956, 2002, all herein incorporated by reference in their entirety).

Interleukin-13 Receptor a2 (IL-13Rα2): IL-13Rα2 is a high affinity receptor for the pleiotropic immune regulatory cytokine interleukin-13 (IL-13) and a known tumor antigen. The significance of IL-13Rα2 expression in cancer is not known and the mechanism of upregulation is still unclear, however, it has been shown that IL-13Rα2 is overexpressed in a variety of human cancers, including malignant glioma, head and neck cancer, Kaposi's sarcoma, renal cell carcinoma, and ovarian carcinoma. Sequences for IL-13Rα2 are known, for example, see NCBI Gene ID: NM_000640.3 for human IL-13Rα2 mRNA, or NCBI Accession NP_000631.1 for human IL-13Rα2 amino acid sequences, all of which are herein incorporated by reference in their entirety.

Isolated or purified: An “isolated” biological component, such as the disclosed immunotoxin, has been substantially separated or purified away from other biological components in the environment (such as a cell) in which the component occurs, e.g., other chromosomal and extra-chromosomal DNA and RNA, proteins and cells. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

The term does not require absolute purity; rather, it is intended as a relative term. Thus, for example, an isolated or purified protein, nucleic acid, or cell preparation is one in which the protein, nucleic acid, or cell is more enriched than the protein, nucleic acid, or cell is in its initial environment. In one embodiment, a preparation is purified such that the protein, nucleic acid, or cell represents at least 50% of the total content of the preparation. A substantially purified protein or nucleic acid is at least 60%, 70%, 80%, 90%, 95% or 98% pure. Thus, in one specific, non-limiting example, a substantially purified protein or nucleic acid is 90% free of other components.

Pharmaceutically Acceptable Carrier: Includes any and all solvents, dispersion media, coatings, antibacterial and antifungal agents, isotonic and absorption delaying agents, and the like, compatible with pharmaceutical administration (see, e.g., Remington: The Science and Practice of Pharmacy, The University of the Sciences in Philadelphia, Editor, Lippincott, Williams, & Wilkins, Philadelphia, PA, 21^{st} Edition, 2005). Examples of such carriers or diluents include, but are not limited to, water, saline, Ringer's solutions, dextrose solution, balanced salt solutions, and 5% human serum albumin. Liposomes and non-aqueous vehicles such as fixed oils may also be used. Supplementary active compounds can also be incorporated into the compositions. Actual methods for preparing administrable compositions include those provided in Remington: The Science and Practice of Pharmacy, The University of the Sciences in Philadelphia, Editor, Lippincott, Williams, & Wilkins, Philadelphia, PA, 21^{st} Edition (2005).

Subject: Living multi-cellular vertebrate organisms, a category that includes human and non-human mammals, including but not limited to non-human primates, rodents, and the like. In specific examples disclosed herein, the subject is human.

Transduced or Transformed: A transformed cell is a cell into which a nucleic acid molecule has been introduced by molecular biology techniques. As used herein, the terms transduction and transformation encompass all techniques by which a nucleic acid molecule might be introduced into such a cell, including transduction or transfection with viral vectors, the use of plasmid vectors, and introduction of DNA by electroporation, lipofection, and particle gun acceleration.

Treating or ameliorating a disease: “Treating” refers to a therapeutic intervention that decreases or inhibits a sign or symptom of a disease or pathological condition after it has begun to develop, such as a reduction in tumor size or tumor burden. “Ameliorating” refers to the reduction in the number or severity of signs or symptoms of a disease, such as cancer.

Vector: A nucleic acid molecule that can be introduced into a host cell (for example, by transfection or transduction), thereby producing a transformed host cell. A vector can include nucleic acid sequences that permit it to replicate in a host cell, such as an origin of replication. A vector can also include one or more selectable marker genes and other genetic elements known in the art. In some examples, the vector is a plasmid. In other examples, the vector is a viral vector. Viral vectors have at least some nucleic acid sequences derived from one or more viruses.

### II. Overview of Several Embodiments

Perineural invasion (PNI) is a prominent feature of certain types of cancers whereby cancer cells invade the surrounding nerves, thus facilitating an alternate route of tumor invasion and metastasis and pain generation. PNI is considered an indicator of aggressive tumor behavior and has been shown to correlate with poor prognosis of patients with solid cancers. Cancer types in which PNI is reported include pancreatic ductal adenocarcinoma (PDAC), head and neck cancer, prostate cancer, colorectal cancer, breast cancer, stomach cancers and cervical cancers. It is likely that other cancers are also associated with PNI. PDAC is one of the most highly aggressive cancers, with high mortality and associated with one of the highest incidences of PNI, which is considered a biomarker of poor prognosis. Here, it is reported that IL-13Rα2 has a role in PNI, thus, immunotoxins specific to IL-13Rα2 may decrease morbidity and cancer associated pain.

Disclosed herein are interleukin-13 receptor a2 (IL-13Rα2) immunotoxins, including a ligand (such as IL-13 or a portion thereof, or an antibody or antibody fragment) specific for IL-13Rα2 linked to a mammalian derived cytotoxic protein. In some examples, the cytotoxic protein is BCL2 associated agonist of cell death (BAD) or Fas-associated death domain (FADD). The immunotoxin may include a linker, for example, a glycine-serine linker between the antibody or antibody fragment and the cytotoxic protein. The disclosed immunotoxins are useful, for example, for treating a tumor or cancer, treating perineural invasion (PNI), or treating pain caused by PNI. In some examples, treatment of a subject with the disclosed immunotoxin treats cancer, reduces PNI, or reduces pain caused by PNI.

### III. IL-13Rα2 Immunotoxins

Disclosed herein are immunotoxins that specifically bind IL-13Rα2. In some examples, the immunotoxin binds a target cell, for example, a cell that expresses IL-13Rα2. In further examples, the immunotoxin binds a cancer or tumor cell expressing IL-13Rα2. The immunotoxin includes a ligand, or an antibody (or antibody fragment), specific for IL-13Rα2 and a cytotoxic protein. In some examples, the immunotoxin is a protein chimera or transcriptional fusion protein including the ligand or antibody (or antibody fragment), and the cytotoxic protein. The immunotoxin may include a linker that attaches the ligand, or the antibody or antibody fragment, and the cytotoxic protein. Suitable linkers include linear flexible peptides, for example, a glycine-serine linker, (Gly-Gly-Gly-Gly-Ser)₃ (SEQ ID NO: 21). Thr-Arg-His-Arg-Gln-Pro-Arg-Gly-Trp-Glu-Gln-Leu (SEQ ID NO: 22), or linkers containing a recognition site for the protease furin. In a specific, non-limiting example, the linker is a glycine-serine linker.

In some embodiments, the cytotoxic protein is of mammalian origin. Examples include, but are not limited to, cytotoxic proteins from human, mouse, non-human primate, mouse, rat, rabbit, pig, goat, sheep, dog, cat, horse, or cow origin. In a specific, non-limiting example, the cytotoxic protein is a human protein. In other embodiments, the cytotoxic protein is of plant or bacterial origin. Suitable cytotoxic proteins of non-mammalian origin include, for example, plant toxins, (e.g., ribotoxins, ricin, saporin, gelonin, and poke weed antiviral protein), bacterial toxins (e.g., anthrax toxin, or diptheria toxin) or chemical toxins (e.g., doxorubicin). In some examples, the cytotoxic protein is a synthetic protein. In some examples, the cytotoxic protein is a ribosylation inactivation protein (RIP), for example, saporin, ricin, riproximin, trichosanthin, gelonium (GAP31), curcin, articulatin-D, moschatin or luffin based RIP immunotoxins. In some examples, the cytotoxic protein is not of bacterial origin, for example, the cytotoxic protein is not a Pseudomonas exotoxin.

In this context, “origin” relates to the sequence of the cytotoxic protein, not how the cytotoxic protein is produced. Thus, for example, a cytotoxic protein of mammalian origin can be produced in any suitable manner (e.g., produced in vitro, or produced in vivo by a bacterial, or any other suitable expression system), so long that the cytotoxic protein shares an amino acid sequence (or at least 70%, at least 80%, at least 90%, at least 95%, at least 98%, at least 99%, or more sequence identity) with a cytotoxic protein of mammalian origin. Similarly, a cytotoxic protein of plant or bacterial origin can be produced in any suitable manner, so long as the cytotoxic protein shares an amino acid sequence (or at least 70%, at least 80%, at least 90%, at least 95%, at least 98%, at least 99%, or more sequence identity) with the corresponding cytotoxic protein of plant or bacterial origin, respectively. The cytotoxic protein need not be a full-length protein. For example, the cytotoxic protein may only include a portion or certain domains of a corresponding full-length cytotoxic protein, so long as the truncated protein retains cytotoxic activity.

The cytotoxic protein includes amino acid sequence variants that retain functionality (e.g., cytotoxicity), for example, the cytotoxic protein has at least 70%, at least 80%, at least 90%, at least 95%, at least 98%, at least 99%, or more sequence identity to a cytotoxic protein and retains cytotoxic activity. In some examples, the cytotoxic protein has at least 95% sequence identity to a cytotoxic protein (e.g., BAD or FADD), and retains cytotoxic activity.

In some examples, the cytotoxic protein is proapoptotic. Examples of proapoptotic proteins include caspases, amyloid-B peptide, B-cell lymphoma/leukemia-2 gene (Bcl-2) proteins, p53, heat shock proteins, or any protein that recruits or otherwise interacts with any of the proapoptotic proteins.

In some examples, the cytotoxic protein contains a death domain motif. Exemplary proapoptotic proteins containing a death domain include: Fas-associated death domain (FADD), tumor necrosis factor receptor type 1-associated DEATH domain (TRADD), cluster of differentiation 95 (CD95; also known as Fas), tumor necrosis factor receptor 1 (TNFR1), death receptor 3 (DR3; also known as TRAMP), death receptor 4 (DR4; also known as TRAIL-R1), and death receptor 5 (DR5; also known as TRAIL-R2). In other examples, the cytotoxic protein contains a BCL2 homology domain 3 (BH3) domain. Exemplary proapoptotic proteins containing a BH3 domain include BH3-interacting domain death agonist (BID), Bcl-2-interacting mediator of cell death (BIM), BCL2 interacting killer (BIK), BCL2 associated agonist of cell death (BAD), Bcl-2-modifying factor (BMF), harakiri (Hrk), Phorbol-12-myristate-13-acetate-induced protein 1 (PMAIP1; also known as NOXA), p53 upregulated modulator of apoptosis (PUMA), B lymphocyte kinase (BLK), BCL2/adenovirus E1B 19 kDa protein-interacting protein 3 (BNIP3), Spike (see, e.g., Mund et al., Spike, a novel BH3-only protein, regulates apoptosis at the endoplasmic reticulum, FASEB J., (6):696-8, 2003), Bcl-2-associated X (BAX; also known as bcl-2-like protein 4), BCL2 Antagonist/Killer 1 (BAK), Bcl-2 related ovarian killer (BOK; also known as MTD), and Bcl-xS. One of ordinary skill in the art can identify other proapoptotic proteins that can be used in the disclosed immunotoxins.

In a non-limiting example, the cytotoxic protein is BAD. The cytotoxic protein can be human BAD, for example, the cytotoxic protein can have at least 70%, at least 80%, at least 90%, at least 95%, at least 98%, at least 99%, or more sequence identity to SEQ ID NO: 5. In some examples, the cytotoxic protein amino acid sequence consists of or includes SEQ ID NO: 5. In some examples, the cytotoxic protein includes or consists of a variant of SEQ ID NO: 5 that retains cytotoxic activity. In some examples, the cytotoxic protein includes or consists of a portion of a BAD protein that retains cytotoxic activity, for example, a portion of SEQ ID NO: 19 that has cytotoxic activity.

In another non-limiting example, the cytotoxic protein is FADD. The cytotoxic protein can be human FADD, for example, the cytotoxic protein can have at least 70%, at least 80%, at least 90%, at least 95%, at least 98%, at least 99%, or more sequence identity to SEQ ID NO: 7. In some examples, the cytotoxic protein amino acid sequence consists of or includes SEQ ID NO: 7. In some examples, the cytotoxic protein includes or consists of a variant of SEQ ID NO: 7 that retains cytotoxic activity. In some examples, the cytotoxic protein includes or consists of a portion of a FADD protein that retains cytotoxic activity, for example, a portion of SEQ ID NO: 20 that has cytotoxic activity.

In some embodiments, the cytotoxic protein has been optimized by introducing amino acid substitutions to increase cytotoxic activity. For example, the cytotoxic protein can have about 1, about 2, about 3, about 4, about 5, about 6, about 7, about 8, about 9, or about 10 substitutions that increase cytotoxic activity. In other examples, the cytotoxic protein has about 1 to 2, 1 to 3, 1 to 4, 1 to 5, 1 to 6, 1 to 7, 1 to 8, 1 to 9, 1 to 10, 2 to 10, 3 to 10, 4 to 10, 5 to 10, 6 to 10, 7 to 10, 8 to 10, 9 to 10, 2 to 4, 2 to 6, 2 to 8, 4 to 6, or 4 to 8, substitutions. In some examples, one or more serine (S) residues are substituted with alanine (A) or aspartic acid (D). In some examples, one or more of the following substitutions are made in BAD: (1) S75A, (2) S99A, (3) S118A, or (4) S134A. In some examples, the following substitution combinations are made in BAD: (1) S75A+S99A, (2) S75A+S99A+S118A, or (3) S75A+S99A+S118A and S134A. In other examples, one or more of the following substitutions are made in FADD: (1) S194A, (2) S203A, (3) S194D, or (4) S203D. In specific, non-limiting examples, the following substitution combinations are made in FADD: (1) S194A and S203A, or (2) S194D and S203D. The residue numbering provided above is based on full length BAD (e.g., SEQ ID NO: 19) or full length FADD (e.g., SEQ ID NO: 20), respectively. In some examples, the amino acid substitutions increase cytotoxic activity of the cytotoxic protein, for example, by at least 10%, at least 20%, at least 30%, at least 40%, at least 50%, at least 60%, at least 70%, at least 80%, at least 90%, at least 100%, at least 150%, at least 200%, at least 300%, at least 400%, at least 500%, or more.

In some embodiments, the immunotoxin includes a ligand that specifically binds IL-13Rα2 and the cytotoxic protein. A ligand that “specifically binds” IL-13Rα2 is a ligand with high affinity to IL-13Rα2 and does not significantly bind to other receptors or proteins. In some examples, the ligand is IL-13, such as human IL-13 (e.g., matured human IL-13), or a portion thereof that specifically binds IL-13Rα2. In some examples, IL-13 includes a signal sequence. In other examples, IL-13 does not include a signal sequence. In some examples, the ligand has at least 70%, at least 80%, at least 90%, at least 95%, at least 98%, at least 99%, or more sequence identity to SEQ ID NO: 9. In some examples, the ligand amino acid sequence consists of or includes SEQ ID NO: 9. In some examples, the ligand has at least 70%, at least 80%, at least 90%, at least 95%, at least 98%, at least 99%, or more sequence identity to a contiguous portion of SEQ ID NO: 23. In some examples, the continuous portion of SEQ ID NO: 23 includes about 50 amino acids of SEQ ID NO: 23, for example, about 55 amino acids, about 60 amino acids, about 65 amino acids, about 70 amino acids, about 75 amino acids, about 80 amino acids, about 90 amino acids, about 100 amino acids, about 105 amino acids, about 110 amino acids, about 115 amino acids, about 120 amino acids, about 125 amino acids, or about 130 amino acids of SEQ ID NO: 23. In a specific, non-limiting example, the ligand includes about 115 contiguous amino acids of SEQ ID NO: 23 and has at least 95% sequence identity to SEQ ID NO: 23. In other examples, the ligand amino acid sequence consists of or includes SEQ ID NO: 23. In further examples, the ligand is a circularly permuted IL-13 (cpIL-13) or a portion thereof that specifically binds IL-13Rα2. In some examples, the ligand has at least 70%, at least 80%, at least 90%, at least 95%, at least 98%, at least 99%, or more sequence identity to cpIL-13. The circularly permuted IL-13 (cpIL-13) and reference sequence have been described, for example, in U.S. Pat. No. 6,518,061. In some examples, the ligand is fused (for example, covalently linked) to the cytotoxic protein. In some examples, the ligand and cytotoxic protein are transcriptionally fused (e.g., expressed from the same transcript).

In some embodiments, the immunotoxin includes an antibody or antibody fragment specific for IL-13Rα2 and the cytotoxic protein. An antibody that “specifically binds” an antigen (such as IL-13Rα2) is an antibody that binds the antigen with high affinity and does not significantly bind other unrelated antigens. In some embodiments, the antibody or antibody fragment includes the CDR sequences provided in Table 1. In some examples, the antibody or antibody fragment binds IL-13Rα2 and includes the variable heavy chain (V_{H}) domain complementarity determining region 1 (CDR1), CDR2, and CDR3 amino acid sequences of SEQ ID NO: 11, and the variable light chain (V_{L}) domain complementarity determining region 1 (CDR1), CDR2, and CDR3 of SEQ ID NO: 11, respectively. In some examples, the antibody or antibody fragment includes an amino acid sequence having at least 90% sequence identity (for example, at least 91%, at least 92%, at least 93%, at least 94%, at least 95%, at least 96%, at least 97%, at least 98% or at least 99% sequence identity) to the V_{H} and V_{L} domains of SEQ ID NO: 11 (e.g., amino acids 1-118 for V_{H} and amino acids 134-239 for V_{L}.). In some examples, the antibody or antibody fragment includes a V_{H} domain including or consisting of amino acids 1-118 of SEQ ID NO: 11 and includes a V_{L} domain including or consisting of amino acids 134-239 of SEQ ID NO: 11.

In some embodiments, the antigen binding fragment is an scFv that includes each of the CDR amino acid sequences provided in Table 1. In some examples, the scFv includes each of the CDR amino acid sequences provided in Table 1 and has at least 90% sequence identity (for example, at least 91%, at least 92%, at least 93%, at least 94%, at least 95%, at least 96%, at least 97%, at least 98% or at least 99% sequence identity) to SEQ ID NO: 11. In further examples, the scFv includes or consists of SEQ ID NO: 11.

TABLE 1
Location of the CDRs in the exemplary
IL-13Ra2 scFv sequence

| | Amino Acid Sequence | Nucleic Acid Sequence |
|---|---|---|
| | (Position in | (Position in |
| CDR | SEQ ID NO: 11) | SEQ ID NO: 12) |
| VH | SYAMH | TCCTATGCCATGCAC |
| CDR1 | (31-35) | (90-104) |
| VH | WINAGNGNTKYSQKFQG | TGGATCAACGCCGGCAACGGC |
| CDR2 | (50-66) | AATACAAAGTACTCTCAGAAG |
| | | TTTCAGGGC |
| | | (147-197) |
| VH | MNHMIPLKA | ATGAATCACATGATCCCACTG |
| CDR3 | (99-107) | AAGGCA |
| | | (294-320) |
| VL | RASQGIRNDLG | CGGGCCAGCCAGGGCATCAGA |
| CDR1 | (157-167) | AACGATCTGGGC |
| | | (468-500) |
| VL | AASSLQS | GCAGCAAGCTCCCTGCAGTCT |
| CDR2 | (183-189) | (546-566) |
| VL | LQMYNYRT | CTGCAGATGTACAATTATAGA |
| CDR3 | (222-229) | ACA |
| | | (663-686) |

In some embodiments, the immunotoxin includes an IL-13 ligand and BAD (or a portion of BAD that retains cytotoxic activity). In some examples the immunotoxin is a chimera including an IL-13 ligand and BAD (or a portion of BAD that retains cytotoxic activity). In some examples, a linker is included between the IL-13 ligand and BAD (or a portion of BAD that retains cytotoxic activity). In some examples, the immunotoxin has at least 70%, at least 80%, at least 90%, at least 95%, at least 98%, at least 99%, or 100% sequence identity to SEQ ID NO: 1. In some examples, the immunotoxin consists of or includes SEQ ID NO: 1.

In further embodiments, the immunotoxin includes an IL-13 ligand and FADD (or a portion of FADD that retains cytotoxic activity). In some examples the immunotoxin is a chimera including an IL-13 ligand and FADD (or a portion of FADD that retains cytotoxic activity). In some examples, a linker is included between the IL-13 ligand and FADD (or a portion of FADD that retains cytotoxic activity). In some examples, the immunotoxin has at least 70%, at least 80%, at least 90%, at least 95%, at least 98%, at least 99%, or 100% sequence identity to SEQ ID NO: 3. In some examples, the immunotoxin consists of or includes SEQ ID NO: 3.

Also provided are functional variants of the immunotoxins described herein, which retain the biological activity of the immunotoxin of which it is a variant (e.g., cytotoxicity). The functional variant can be at least about 80%, about 85%, about 90%, about 91%, about 92%, about 93%, about 94%, about 95%, about 96%, about 97%, about 98%, about 99%, or more identical in amino acid sequence to the sequence of a parent immunotoxin or component thereof, such as a parent ligand, antibody (or antibody fragment), or cytotoxic protein. In some examples, the functional variant includes the amino acid sequence of the parent immunotoxin or component thereof, with at least one conservative amino acid substitution. For example, no more than about 1, about 2, about 3, about 4, about 5, about 6, about 7, about 8, about 9, or about 10 conservative substitutions. In other examples, the functional variant has about 1 to 2, 1 to 3, 1 to 4, 1 to 5, 1 to 6, 1 to 7, 1 to 8, 1 to 9, 1 to 10, 2 to 10, 3 to 10, 4 to 10, 5 to 10, 6 to 10, 7 to 10, 8 to 10, 9 to 10, 2 to 4, 2 to 6, 2 to 8, 4 to 6, or 4 to 8, conservative substitutions. In other examples, the functional variant includes the amino acid sequence of the parent immunotoxin with about 1 to about 4 non-conservative amino acid substitutions. In some examples the functional variant includes no more than about 1, about 2, about 3, about 4, about 5, about 6, about 7, about 8, about 9, or about 10 non-conservative substitutions. In further examples, the functional variant has about 1 to 2, 1 to 3, 1 to 4, 1 to 5, 1 to 6, 1 to 7, 1 to 8, 1 to 9, 1 to 10, 2 to 10, 3 to 10, 4 to 10, 5 to 10, 6 to 10, 7 to 10, 8 to 10, 9 to 10, 2 to 4, 2 to 6, 2 to 8, 4 to 6, or 4 to 8, non-conservative substitutions. In this case, the non-conservative amino acid substitution does not interfere with or inhibit the biological activity of the functional variant. The non-conservative amino acid substitution may enhance the biological activity (e.g., cytotoxic activity, or binding specificity for IL-13Rα2) of the functional variant, such that the biological activity of the functional variant is increased as compared to the parent immunotoxin.

The immunotoxin can, in some examples, include one or more synthetic amino acids in place of one or more naturally occurring amino acids. Such synthetic amino acids include, for example, aminocyclohexane carboxylic acid, norleucine, α-amino n-decanoic acid, homoserine, S-acetylaminomethyl-cysteine, trans-3-and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, oc-aminocycloheptane carboxylic acid, -(2-amino-2-norbornane)-carboxylic acid, γ-diaminobutyric acid, α,β-diaminopropionic acid, homophenylalanine, and α-tert-butylglycine. The immunotoxins may be glycosylated, amidated, carboxylated, phosphorylated, esterified, N-acylated, cyclized via, e.g., a disulfide bridge, or converted into an acid addition salt and/or optionally dimerized or polymerized, or conjugated.

Also provided herein are nucleic acid molecules encoding the disclosed immunotoxins. In some examples, the nucleic acid molecule can be codon optimized for expression in a prokaryotic (e.g., bacteria, archaea) or eukaryotic (e.g., fungi (e.g., yeast), insect, animal, plant) organism. In some examples, the nucleic acid molecule is codon optimized for expression in a particular organism or system, such as E. coli, S. cerevisiae, or mammalian cell lines. In some examples, the nucleic acid molecule is codon optimized for expression in a prokaryotic cell, such as E. coli (e.g., BL21(DE3) cells). In other examples, the nucleic acid molecule is codon optimized for expression in a eukaryotic cell, for example, Chinese hamster ovarian (CHO), HeLa or myeloma cell lines.

In some embodiments, the nucleic acid molecule encodes the immunotoxin including an IL-13 ligand. In some examples, the nucleic acid molecule encodes the immunotoxin including an IL-13 ligand and BAD (or a portion thereof that retains cytotoxic activity). In some examples, the nucleic acid molecule encodes a protein having at least 70%, at least 80%, at least 90%, at least 95%, at least 98%, at least 99%, or 100% sequence identity to SEQ ID NO: 1. In some examples, the nucleic acid molecule encodes a protein that includes or consists of SEQ ID NO: 1. In further examples, the nucleic acid molecule has at least 70%, at least 80%, at least 90%, at least 95%, at least 98%, at least 99%, or 100% sequence identity to SEQ ID NO: 2. In some examples, the nucleic acid molecule includes or consists of SEQ ID NO: 2.

In some examples, the nucleic acid molecule encodes an immunotoxin including an IL-13 ligand and FADD (or a portion thereof that retains cytotoxic activity). In some examples, the nucleic acid molecule encodes a protein having at least 70%, at least 80%, at least 90%, at least 95%, at least 98%, at least 99%, or 100% sequence identity to SEQ ID NO: 3. In some examples, the nucleic acid molecule encodes a protein that includes or consists of SEQ ID NO: 3. In further examples, the nucleic acid molecule has at least 70%, at least 80%, at least 90%, at least 95%, at least 98%, at least 99%, or 100% sequence identity to SEQ ID NO: 4. In specific examples, the nucleic acid molecule includes or consists of SEQ ID NO: 4.

In additional embodiments, the nucleic acid molecule encodes an immunotoxin including an antibody or antibody fragment that specifically binds IL-13Rα2, and the nucleic acid encodes each of the CDR sequences provided in Table 1. In some examples, the nucleic acid encodes each of the CDR sequences provided in Table 1 and encodes an amino acid sequence having at least 70%, at least 80%, at least 90%, at least 95%, at least 98%, at least 99%, or 100% sequence identity to SEQ ID NO: 11. In some examples, the nucleic acid encodes each of the CDR sequences provided in Table 1 and encodes an amino acid sequence having at least 90% sequence identity to SEQ ID NO: 11. In further examples, the nucleic acid encodes each of the CDR sequences provided in Table 1, and has at least 90% sequence identity (for example, at least 91%, at least 92%, at least 93%, at least 94%, at least 95%, at least 96%, at least 97%, at least 98% or at least 99% sequence identity) to the nucleotide sequence of SEQ ID NO: 12. In some examples, the nucleic acid includes or consists of SEQ ID NO: 12.

The disclosed nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements). Nucleic acids can also be prepared by amplification methods. Amplification methods include the polymerase chain reaction (PCR), the ligase chain reaction (LCR), the transcription-based amplification system (TAS), the self-sustained sequence replication system (3SR), and the Qβ replicase amplification system (QB). The disclosed nucleic acids can be prepared by direct chemical synthesis by standard methods. Chemical synthesis produces a single stranded oligonucleotide. This can be converted into double stranded DNA by hybridization with a complementary sequence or by polymerization with a DNA polymerase using the single strand as a template. A wide variety of cloning, in vitro amplification and synthesis methodologies have been described.

Modifications can be made to the disclosed nucleic acids without diminishing biological activity of the encoded immunotoxin. For example, modifications can be made to facilitate cloning, expression, or recovery of the immunotoxin. Such modifications include, for example, addition of termination codons, sequences to create restriction sites, sequences to add a methionine to provide an initiation site, or the addition of other amino acids (e.g., poly His or a cleavable tag) to aid in purification steps.

Also provided are vectors that include the disclosed nucleic acids encoding the immunotoxin. In some embodiments, the disclosed nucleic acids are included in a vector (e.g., viral vector, plasmid, or other vehicle) for expression in a host cell. In some examples, the vector includes a promoter operably linked to the disclosed nucleic acid molecule. Additional expression control sequences, such as one or more enhancers, transcription and/or translation terminators, and initiation sequences can also be included in the expression vector.

In some embodiments, the vector is a viral vector. Exemplary viral vectors include, but are not limited to, polyoma, SV40, adenovirus, vaccinia virus, adeno-associated virus, herpes viruses including HSV and EBV, Sindbis viruses, alphaviruses and retroviruses of avian, murine, or human origin. Baculovirus (Autographa californica multinuclear polyhedrosis virus; AcMNPV) vectors can also be used. Other suitable vectors include orthopox vectors, avipox vectors, fowlpox vectors, capripox vectors, suipox vectors, lentiviral vectors, alpha virus vectors, and poliovirus vectors. Specific exemplary vectors are poxvirus vectors such as vaccinia virus, fowlpox virus and a highly attenuated vaccinia virus (MVA), adenovirus, baculovirus and the like. Pox viruses of use include orthopox, suipox, avipox, and capripox virus. Orthopox include vaccinia, ectromelia, and raccoon pox. One example of an orthopox of use is vaccinia. Avipox includes fowlpox, canary pox and pigeon pox. Capripox include goatpox and sheeppox. In one example, the suipox is swinepox. Other viral vectors that can be used include other DNA viruses such as herpes virus and adenoviruses, and RNA viruses such as retroviruses and polio.

In some embodiments, the vector is a plasmid. In some examples, the plasmid is a vector for bacterial expression. In some examples, the vector is suitable for expression in E. coli (e.g., BL21). Exemplary plasmid vectors include pBluescript, pET, pGEX, pMAL, pQE, pGS, pETDuet, pCDFDuet, and pCOLADuet vectors. In some examples, the vector is a pET vector, for example, pET3a, pET3b, pET3c, pET3d, pET9a, pET11a, pET11b, pET11c, pET11d, pET14b, pET15b, pET16b, pET17b, pET19b, pET20b, pET21a, pET21b, pET21d, pET22b, pET23a, pET24a, pET24b, pET24c, pET24d, pET25b, pET26b, pET27b, pET28a, pET28b, pET28c, pET29a, pET29b, pET29c, pET301, pET30b, pET30c, pET31b, pET32a, pET32b, pET41a, pET41b, pET41c, pET42b, pET42c, pET43.1a, pET43.1b, pET50b, pET51b, or pET52b. In some examples, the vector is pET24a.

Biologically functional viral and plasmid DNA vectors that are capable of expression in a cell, for example, in E. coli, are known and a suitable vector can be identified. In some examples, the vector includes a selectable marker (such as an antibiotic resistance gene, for example puromycin) or a reporter gene (such as green fluorescent protein (GFP)). In other examples, a selectable marker and/or reporter is not included in the vector.

In some examples, the vector includes a nucleic acid having at least 90% sequence identity (for example, at least 91%, at least 92%, at least 93%, at least 94%, at least 95%, at least 96%, at least 97%, at least 98% at least 99%, or 100% identity) to SEQ ID NO: 2. In some examples, the vector includes a nucleic acid including or consisting of SEQ ID NO: 2. In further examples, the vector includes a nucleic acid encoding an amino acid sequence having at least 90% sequence identity (for example, at least 91%, at least 92%, at least 93%, at least 94%, at least 95%, at least 96%, at least 97%, at least 98% at least 99%, or 100% identity) to SEQ ID NO: 1. In some examples, the vector includes a nucleic acid encoding an amino acid sequence including or consisting of SEQ ID NO: 1. In other examples, the vector includes a nucleic acid having at least 90% sequence identity (for example, at least 91%, at least 92%, at least 93%, at least 94%, at least 95%, at least 96%, at least 97%, at least 98% at least 99%, or 100% identity) to SEQ ID NO: 4. In some examples, the vector includes a nucleic acid including or consisting of SEQ ID NO: 4. In further examples, the vector includes a nucleic acid encoding an amino acid sequence having at least 90% sequence identity (for example, at least 91%, at least 92%, at least 93%, at least 94%, at least 95%, at least 96%, at least 97%, at least 98% at least 99%, or 100% identity) to SEQ ID NO: 3. In some examples, the vector includes a nucleic acid encoding an amino acid sequence including or consisting of SEQ ID NO: 3.

Also disclosed herein are host cells including the disclosed nucleic acids or vectors encoding the immunotoxin. The nucleic acids or vectors can be expressed in vitro by DNA transfer into a suitable host cell. The host cell may be prokaryotic or eukaryotic. In some examples, the host cell is E. coli. In a specific, non-limiting example, the host cell is E. coli strain BL21(DE3)pLysS. Numerous expression systems are available for expression of proteins in host cells, including, E. coli (and other bacterial hosts), yeast, and various higher eukaryotic cells such as COS, Chinese hamster ovarian (CHO), HeLa, and myeloma cell lines, for example, to be used to express the disclosed immunotoxin. Methods of stable transfer, meaning that the foreign DNA is continuously maintained in the host, may be used.

To obtain optimal expression of the disclosed nucleic acids, vectors can contain, for example, a strong promoter to direct transcription, a ribosome binding site for translational initiation (e.g., internal ribosomal binding sequences), and a transcription/translation terminator can be used. For expression in E. coli, a promoter, such as the T7, trp, lac, or lambda promoters, a ribosome binding site, and a transcription termination signal can be used. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). Additional operational elements include, but are not limited to, leader sequence, termination codons, polyadenylation signals and any other sequences necessary for the appropriate transcription and subsequent translation of the nucleic acid sequence.

A host cell can be transformed or transfected by any suitable method, such as chemical transformation or electroporation for E. coli, and electroporation or lipofection for mammalian cells. Cells transformed by the disclosed vectors can be selected by resistance to antibiotics conferred by genes contained in the vector (e.g., amp^{r}, gmr, kan^{r}, neo, rif^{r}, cmlA, and hyg genes).

When the disclosed nucleic acid (or nucleic acid included in a vector) is expressed in a host cell, the encoded immunotoxin can be purified according to standard procedures in the art, including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). When a tag is present (e.g., a cleavable/removable tag), the immunotoxin can be isolated using a binding affinity column or beads specific to the tag (e.g., Ni-NTA, anti-GFP). Tags (when used) can be removed from the immunotoxin, or may be retained. In some examples, the immunotoxin is isolated using Fast Protein Liquid Chromatography with Q sepharose, mono Q and sephacryl S-100 gel exclusion columns. The purified immunotoxin need not be 100% pure, but is substantially pure, for example, at least 60%, 70%, 80%, 90%, 95% or 98% pure. In some examples, the purified immunotoxin is 90% free of other components (e.g., other proteins or cellular debris). In addition to the examples provided herein, methods for expression of proteins and/or refolding to an appropriate active form from mammalian cells and bacteria, such as E. coli, have been described. See, e.g., Basic methods in Protein Purification and Analysis: A Laboratory Manual, New York: Cold Spring Harbor Laboratory Press, 2009.

### IV. Methods of Treatment

Also disclosed herein are methods of treating an IL-13Rα2-expressing tumor or cancer, reducing perineural invasion (PNI) of an IL-13Rα2-expressing tumor or cancer, as well as methods of reducing pain resulting from PNI of an IL-13Rα2-expressing tumor or cancer, in a subject by administering an effective amount of a disclosed immunotoxin, an IL-13-PE immunotoxin, or both. IL-13-PE immunotoxins have been previously described, for example, in Kioi et al., Mol. Cancer Ther. 7(6): 1579-1587, 2008 and U.S. Pat. No. 6,518,061, both herein incorporated by reference in their entirety. In specific, non-limiting examples, an effective amount of the disclosed immunotoxin is administered. In some examples, an IL-13-PE immunotoxin is not administered with the disclosed immunotoxin.

In some examples, the subject has a cancer that expresses IL-13Rα2. In some examples, the subject has a cancer that over-expresses IL-13Rα2, for example, relative to a non-cancerous cell. In some examples, the subject has a solid tumor. In specific non-limiting examples, the subject has pancreatic cancer, stomach cancer, colorectal cancer, kidney cancer, glioblastoma (or other malignant glioma), head and neck cancer, squamous cell carcinoma of the skin, biliary tract tumor, cholangiocarcinoma, vulvar carcinoma, oral cancer, ovarian cancer, uterine cancer, cervical cancer, prostate cancer, breast cancer, melanoma, non-small cell lung cancer (NSCLC), renal cell carcinoma, Kaposi's sarcoma, or adrenocortical cancer.

In some examples, administration of an effective amount of a disclosed immunotoxin or IL-13-PE immunotoxin treats the cancer, PNI invasion, or pain caused by PNI. For example, in some examples, the effective amount of the immunotoxin is an amount sufficient to prevent, treat, reduce, and/or ameliorate one or more signs or symptoms of cancer in the subject, for example, an amount sufficient for specific killing of a cancer or a tumor cell. In some examples, the effective amount is an amount sufficient to reduce tumor size or tumor load in the subject by at least 10%, at least 20%, at least 30%, at least 40%, at least 50%, at least 60%, at least 70%, at least 80%, at least 90%, or 100%, as compared to a suitable control. In some examples, the effective amount is an amount sufficient to inhibit or slow metastasis in the subject. In some examples, the effective amount inhibits or decreases tumor spread in the subject by at least 10%, at least 20%, at least 30%, at least 40%, at least 50%, at least 60%, at least 70%, at least 80%, at least 90%, or 100% as compared to a suitable control. In some examples, the effective amount is an amount that increases life expectancy of the subject, for example, by at least 10%, at least 20%, at least 30%, at least 40%, at least 50%, at least 60%, at least 70%, at least 80%, at least 90%, at least 100%, at least 200%, at least 400%, or more as compared to a suitable control. In other examples, the effective amount is an amount sufficient to reduce tumor density in the subject, for example, by at least 10%, at least 20%, at least 30%, at least 40%, at least 50%, at least 60%, at least 70%, at least 80%, at least 90%, or 100% as compared to a suitable control. In further examples, the effective amount is an amount sufficient to target and eliminate tumor cells, for example, by at least 10%, at least 20%, at least 30%, at least 40%, at least 50%, at least 60%, at least 70%, at least 80%, at least 90%, or 100% in the subject as compared to a suitable control. In some examples, the effective amount reduces PNI invasion, for example, by reducing invasion of IL-13Rα2-expressing tumor cells by at least 10%, at least 20%, at least 30%, at least 40%, at least 50%, at least 60%, at least 70%, at least 80%, at least 90%, or 100% compared to a suitable control. In some examples, the effective amount reduces nerve pain in the subject, for example, by at least 10%, at least 20%, at least 30%, at least 40%, at least 50%, at least 60%, at least 70%, at least 80%, at least 90%, or 100% compared to a suitable control. Nerve pain can be assessed qualitatively, for example, by the subject reporting a reduction in perceived pain. Examples of suitable controls include baseline measurements of the subject prior to treatment, untreated subjects, or subjects not receiving the immunotoxin or IL-13-PE immunotoxin (e.g., subjects receiving other agents or alternative treatments). One of ordinary skill in the art can select suitable controls depending on the situation.

In some examples, the methods include administering to the subject a composition including a disclosed immunotoxin (or IL-13-PE immunotoxin) and a pharmaceutically acceptable carrier. A “pharmaceutically acceptable carrier” includes any and all solvents, dispersion media, coatings, antibacterial and antifungal agents, isotonic and absorption delaying agents, and the like, compatible with pharmaceutical administration (see, e.g., Remington: The Science and Practice of Pharmacy, The University of the Sciences in Philadelphia, Editor, Lippincott, Williams, & Wilkins, Philadelphia, PA, 21^{st} Edition, 2005). Examples of such carriers or diluents include, but are not limited to, water, saline, Ringer's solutions, dextrose solution, balanced salt solutions, and 5% human serum albumin. Liposomes and non-aqueous vehicles such as fixed oils may also be used. Supplementary active compounds can also be incorporated into the compositions. Actual methods for preparing administrable compositions include those provided in Remington: The Science and Practice of Pharmacy, The University of the Sciences in Philadelphia, Editor, Lippincott, Williams, & Wilkins, Philadelphia, PA, 21^{st} Edition (2005).

In some embodiments, about 0.1 to 100 μg/kg body weight of a disclosed immunotoxin or IL-13-PE immunotoxin is administered to the subject per day (for example, about 0.1 to 10 μg/kg, 0.1 to 15 μg/kg, 0.1 to 20 μg/kg, 0.1 to 30 μg/kg, 0.1 to 40 μg/kg, 0.1 to 50 μg/kg, 0.1 to 60 μg/kg, 0.1 to 70 μg/kg, 0.1 to 80 μg/kg, 0.1 to 90 μg/kg, 1 to 10 μg/kg, 1 to 20 μg/kg, 1 to 30 μg/kg, 1 to 40 μg/kg, 1 to 50 μg/kg, 1 to 60 μg/kg, 1 to 70 μg/kg, 1 to 80 μg/kg, 1 to 90 μg/kg, 1 to 100 μg/kg, 10 to 100 μg/kg, 20 to 100 μg/kg, 30 to 100 μg/kg, 40 to 100 μg/kg, 50 to 100 μg/kg, 60 to 100 μg/kg, 70 to 100 μg/kg, 80 to 100 μg/kg, 90 to 100 μg/kg, 10 to 90 μg/kg, 10 to 80 μg/kg, 10 to 70 μg/kg, 10 to 60 μg/kg, 10 to 50 μg/kg, 10 to 40 μg/kg, 10 to 30 μg/kg, 10 to 20 μg/kg, 20 to 90 μg/kg, 20 to 80 μg/kg, 20 to 70 μg/kg, 20 to 60 μg/kg, 20 to 50 μg/kg, 20 to 40 μg/kg, or 20 to 30 μg/kg per day). In some examples, about 0.1 to 5 μg/kg is administered per day, for example, about 0.1 to 4 μg/kg, about 0.1 to 3 μg/kg, about 0.1 to 2 μg/kg, about 0.1 to 1 μg/kg, about 0.5 to 5 μg/kg, about 1 to 5 μg/kg, about 2 to 5 μg/kg, about 3 to 5 μg/kg, about 4 to 5 μg/kg, about 0.5 to 4 μg/kg, about 1 to 4 μg/kg, about 1 to 3 μg/kg, about 2 to 5 μg/kg. In a specific example, about 1 to 2 μg/kg is administered per day. In additional examples, about 60 to 100 μg/kg, 70 to 100 μg/kg, 80 to 100 μg/kg, 90 to 100 μg/kg, 50 to 90 μg/kg, 50 to 80 μg/kg, 50 to 70 μg/kg, 50 to 60 μg/kg body weight is administered per day. In a specific, non-limiting example, about 50 to 100 μg/kg body weight is administered per day. Higher concentrations may be used for local delivery near a tumor. Appropriate dosage can be determined by a skilled clinician based on factors such as the subject (e.g., species, weight), the condition being treated, severity of the condition, and other factors.

In further examples, about 0.1 to 100 mg of a disclosed immunotoxin or IL-13-PE immunotoxin is administered to the subject per day (for example, about 0.1 to 10 mg, 0.1 to 11 mg, 0.1 to 12 mg, 0.1 to 13 mg, 0.1 to 14 mg, 0.1 to 15 mg, 0.1 to 20 mg, 0.1 to 30 mg, 0.1 to 40 mg, 0.1 to 50 mg, 0.1 to 60 mg, 0.1 to 70 mg, 0.1 to 80 mg, 0.1 to 90 mg, 1 to 10 mg, 1 to 20 mg, 1 to 30 mg, 1 to 40 mg, 1 to 50 mg, 1 to 60 mg, 1 to 70 mg, 1 to 80 mg, 1 to 90 mg, 1 to 100 mg, 10 to 100 mg, 20 to 100 mg, 30 to 100 mg, 40 to 100 mg, 50 to 100 mg, 60 to 100 mg, 70 to 100 mg, 80 to 100 mg, 90 to 100 mg, 10 to 90 mg, 10 to 80 m, 10 to 70 mg, 10 to 60 mg, 10 to 50 mg, 10 to 40 mg, 10 to 30 mg, 10 to 20 mg, 20 to 90 mg, 20 to 80 mg, 20 to 70 mg, 20 to 60 mg, 20 to 50 mg, 20 to 40 mg, or 20 to 30 mg). In some examples about 0.1 to 10 mg is administered per day. In some examples, the amount is about 0.1 to 1 mg, 0.1 to 2 mg, 0.1 to 3 mg, 0.1 to 4 mg, 0.1 to 5 mg, 0.1 to 6 mg, 0.1 to 7 mg, 0.1 to 8 mg, 0.1 to 9 mg, 0.1 to 10 mg, 0.2 to 10 mg, 0.3 to 10 mg, 0.4 to 10 mg, 0.5 to 10 mg, 0.6 to 10 mg, 0.7 to 10 mg, 0.8 to 10 mg, 0.9 to 10 mg, 1 to 10 mg, 2 to 10 mg, 3 to mg, 4 to 10 mg, 5 to 10 mg, 6 to 10 mg, 7 to 10 mg, 8 to 10 mg, 9 to 10 mg, 0.1 to 5 mg, 0.1 to 8 mg, 0.5 to 5 mg, 0.5 to 8 mg, 0.5 to 10 mg, 1 to 5 mg, 1 to 8 mg, 2 to 5 mg, 2 to 8 mg, 4 to 5 mg, or 4 to 8 mg per day.

Multiple doses of the immunotoxin can be administered to a subject. For example, the immunotoxin can be administered daily, every other day, twice per week, weekly, every other week, every three weeks, monthly, or less frequently. In some examples, the immunotoxin is administered on days 1, 3, and 5, of a 4-week cycle. Multiple cycles may be administered to the subject, for example, about 2 cycles, 3 cycles, 4 cycles, 5 cycles, 6 cycles, or more. A skilled clinician can select an administration schedule based on the subject, the condition being treated, the previous treatment history, and other factors.

Administration of a disclosed immunotoxin, or IL-13-PE immunotoxin, can be local or systemic. Exemplary routes of administration include, but are not limited to, oral, injection (such as subcutaneous, intramuscular, intradermal, intraperitoneal, intravenous), sublingual, rectal, transdermal (for example, topical), intranasal, vaginal, and inhalation routes. The immunotoxin is typically administered parenterally, for example intravenously; however, injection or infusion into a tumor or close to a tumor (local administration) or administration to the peritoneal cavity can also be used. Appropriate routes of administration can be determined by a skilled clinician based on factors such as the subject, the condition being treated, and other factors.

In some embodiments, the subject receives an additional treatment, such as one or more of surgery, radiation, chemotherapy, biologic therapy, additional immunotherapy, or other cancer therapy. Exemplary chemotherapeutic agents include (but are not limited to) alkylating agents, such as nitrogen mustards (such as mechlorethamine, cyclophosphamide, melphalan, uracil mustard or chlorambucil), alkyl sulfonates (such as busulfan), nitrosoureas (such as carmustine, lomustine, semustine, streptozocin, or dacarbazine); antimetabolites such as folic acid analogs (such as methotrexate), pyrimidine analogs (such as 5-FU or cytarabine), and purine analogs, such as mercaptopurine or thioguanine; or natural products, for example vinca alkaloids (such as vinblastine, vincristine, or vindesine), epipodophyllotoxins (such as etoposide or teniposide), antibiotics (such as dactinomycin, daunorubicin, doxorubicin, bleomycin, plicamycin, or mitocycin C), and enzymes (such as L-asparaginase). Additional agents include platinum coordination complexes (such as cis-diamine-dichloroplatinum II, also known as cisplatin), substituted ureas (such as hydroxyurea), methyl hydrazine derivatives (such as procarbazine), and adrenocrotical suppressants (such as mitotane and aminoglutethimide); hormones and antagonists, such as adrenocorticosteroids (such as prednisone), progestins (such as hydroxyprogesterone caproate, medroxyprogesterone acetate, and magestrol acetate), estrogens (such as diethylstilbestrol and ethinyl estradiol), antiestrogens (such as tamoxifen), and androgens (such as testosterone proprionate and fluoxymesterone). Examples of the most commonly used chemotherapy drugs include adriamycin, melphalan (Alkeran®) Ara-C(cytarabine), carmustine, busulfan, lomustine, carboplatinum, cisplatinum, cyclophosphamide (Cytoxan®), daunorubicin, dacarbazine, 5-fluorouracil, fludarabine, hydroxyurea, idarubicin, ifosfamide, methotrexate, mithramycin, mitomycin, mitoxantrone, nitrogen mustard, paclitaxel (or other taxanes, such as docetaxel), vinblastine, vincristine, VP-16, while newer drugs include gemcitabine (Gemzar®), trastuzumab (Herceptin®), irinotecan (CPT-11), leustatin, navelbine, rituximab (Rituxan®) imatinib (STI-571), Topotecan (Hycamtin®), capecitabine, ibritumomab (Zevalin®), and calcitriol. A skilled clinician can select appropriate additional therapies (from those listed here or other current therapies) for the subject, depending on factors such as the subject, the cancer being treated, treatment history, and other factors. Non-limiting examples of immunotherapy include adoptive cell therapy (ACT), monoclonal antibodies, cancer vaccines, and immune system modulators (e.g., cytokines or immunomodulatory drugs such as thalidomide, lenalidomide, pomalidomide, or imiquimod).

In some embodiments, the subject is administered an additional therapeutic, such as a checkpoint inhibitor (e.g., anti-CTLA-4, anti-PD1, or anti-PDL1), a histone deacetylase (HDAC) inhibitor, a cell cycle or spindle assembly checkpoint inhibitor (e.g., UCN-01, ICP-1, PF00477736, XL9844, PD321852, CEP3891, AZD7762, LY2603618, Gö6976, SCH900776, CCT244747, NU6027, MK-1775, Taxanes, Vinca alkaloids, and MK-1775; see, e.g., Visconti et al., J Exp Clin Cancer Res. 35(1): 153, 2016), adrenomedullin, another immunotoxin, or any combination of two or more thereof. The administration of an additional therapeutic may be before, after, or substantially simultaneously with the administration of the immunotoxin. In some examples, the additional therapeutic is administered substantially simultaneously with the immunotoxin (such as on the same day or within 30 minutes to 6 hours of administration of the immunotoxin). In other examples, the additional therapeutic is administered prior to administering the immunotoxin, for example, at least 1 day, at least 2 days, at least 3 days, at least 4 days, at least 5 days, at least 6 days, at least 7 days, at least 8 days, at least 9 days, at least 12 days, at least 14 days, at least three weeks, at least four weeks, at least one month, or more prior. Multiple doses of the additional therapeutic can be administered to a subject, for example, administered twice daily, once daily, every other day, twice per week, weekly, every other week, every three weeks, monthly, or less frequently. A skilled clinician can select an administration schedule based on the subject, the condition being treated, the previous treatment history, tumor load and type, clinical stage and grade of the disease, overall health of the subject, and other factors.

In some examples, the additional therapeutic increases expression of IL-13Rα2 on the IL-13Rα2-expressing tumor or cancer. For example, it has been reported that treatment with HDAC inhibitors dramatically upregulates IL-13Rα2 in pancreatic cancer cell lines expressing little to no IL-13Rα2. These inhibitors also modestly upregulated IL-13Rα2 in cells expressing higher levels of IL-13Rα2. Upregulation of IL-13Rα2 was found to sensitize pancreatic tumor cells to IL-13-PE (see Fujisawa et al. Journal of Translational Medicine 9:37, 2011). Thus, in some examples, the subject is administered an HDAC inhibitor, such as one or more of Trichostatin A (TSA), suberoylanilide hydroxamic acid (SAHA), sodium butyrate (NaB), SP600125 (Sigma-Aldrich), SR11302 (Tocris Bioscience), or Romidepsin. HDAC inhibitors have been described, for example, see Marks and Jiang, Cell Cycle 2005, 4:549-551; Duvic et al. Blood 109:31-39, 2007; and Fujisawa et al. Journal of Translational Medicine 9:37, 2011). In another example, the subject is administered adrenomedullin, which has also been shown to increase IL-13Rα2 expression (Joshi et al., Cancer Res. 68:9311-9317, 2008).

In some examples, the subject is administered a checkpoint inhibitor. In some examples, the checkpoint inhibitor targets PD-1, PD-L1, CTLA-4, CDK4, and/or CDK6. Exemplary inhibitors include ipilimumab, nivolumab, pembrolizumab, atezolizumab, avelumab, durvalumab, cemiplimab, palbociclib, ribociclib, and abemaciclib. The checkpoint inhibitor may be administered substantially simultaneously with the immunotoxin. In some examples, the checkpoint inhibitor is administered prior to administering the immunotoxin, for example, at least 1 day, at least 2 days, at least 3 days, at least 4 days, at least 5 days, at least 6 days, at least 7 days, at least 8 days, at least 9 days, at least 12 days, at least 14 days, at least three weeks, at least four weeks, at least one month, or more prior. Multiple doses of the checkpoint inhibitor can be administered to the subject, for example, administered twice daily, once daily, every other day, twice per week, weekly, every other week, every three weeks, monthly, or less frequently. A skilled clinician can select an administration schedule based on the subject, the condition being treated, the previous treatment history, tumor load and type, clinical stage and grade of the disease and overall health of the subject, and other factors.

In further examples, the subject is administered another immunotoxin, for example an IL-13 PE immunotoxin in addition to the disclosed immunotoxin. The IL-13-PE immunotoxin can be a recombinant protein including IL-13 conjugated to a truncated Pseudomonas exotoxin (see, e.g., Kioi et al., Mol. Cancer Ther. 7(6):1579-1587, 2008; and U.S. Pat. No. 6,518,061, herein incorporated by reference in their entirety). The IL-13 PE immunotoxin may be administered substantially simultaneously with the immunotoxin. In some examples, the IL-13 PE immunotoxin is administered prior to administering the immunotoxin, for example, at least 1 day, at least 2 days, at least 3 days, at least 4 days, at least 5 days, at least 6 days, at least 7 days, at least 8 days, at least 9 days, at least 12 days, at least 14 days, at least three weeks, at least four weeks, at least one month, or more prior. Multiple doses of the IL-13 PE immunotoxin can be administered to the subject, for example, administered twice daily, once daily, every other day, twice per week, weekly, every other week, every three weeks, monthly, or less frequently. A skilled clinician can select an administration schedule based on the subject, the condition being treated, the previous treatment history, tumor load and type, clinical stage and grade of the disease and overall health of the subject, and other factors.

Also disclosed herein are methods of killing a cancer or tumor cell expressing IL-13Rα2, comprising contacting the cancer or tumor cell with an effective amount of a disclosed immunotoxin, thereby killing the cell. The method can be in vitro, for example, in a cell culture, or the method can be in vivo, for example, in a subject. An appropriate effective amount can be readily determined by one of skill in the art, for example, by performing cytotoxicity assays to determine an amount that induces killing of a cancer or tumor cell.

### EXAMPLES

The following examples are provided to illustrate certain particular features and/or embodiments. These examples should not be construed to limit the disclosure to the particular features or embodiments described.

### Example 1

### Materials and Methods

### Patient and Samples

PDAC samples were obtained from a total of 236 patients who underwent macroscopic curative resection at NTT Medical Center Tokyo (NTT; 107 patients) and Yokohama City University Hospital (YCU; 129 patients) from January 1993 to September 2013. Only patients with PDAC were included while patients with tumor derived from intraductal papillary mucinous neoplasm or other types of cystic lesions were excluded from the study. Patients with uncommon histological tumors, including adeno-squamous carcinoma, mucinous carcinoma, anaplastic carcinoma, undifferentiated carcinoma, acinar cell carcinoma, and neuroendocrine carcinoma were excluded.

The clinical parameters that were analyzed include age, sex, tumor location, tumor size, diabetes before surgery, tumor biomarkers (carcinoembryonic antigen; CEA, and carbohydrate antigen 19-9; CA19-9), and cancer staging as per Union Internationale Contre le Cancer (UICC) (Sobin et al. (2004) Cancer, 100:1106). Pathologic findings of the tumors were evaluated for tumor factor (T), regional lymph node metastasis (N), distant lymph node metastasis (M), invasion to lymph ducts (Ly), to veins (V), to nerve in the pancreas (Ne), to bile duct (CH), to duodenum (DU), to front constructs of pancreas (S), to back constructs of pancreas (RP), to portal vein (PV), to artery (A), to peripancreatic neuroplexus (PL), and to other organs (OO). The method of surgery was determined by the location of the tumor. 161 patients with a tumor in the head of the pancreas underwent pancreaticoduodenectomy, and 75 patients with a tumor in the body and tail underwent distal pancreatectomy. No patients underwent total or subtotal pancreatectomy. PDAC tumors from patients were surgically resected and followed up periodically for post-surgical care. They were maintained with a standard chemotherapy regimen of gemcitabine as an adjuvant chemotherapy (1000 mg/m2) with intravenous infusion given once a week for 6 months. In case of tumor recurrence, 100 mg/m2 of tegafur/gimeracil/oteracil (S-1) was orally administered to the patients twice a day.

### Immunohistochemistry (IHC)

PDAC tissue sections were prepared in 4-μm-thick section on a poly-L-Lysine coated glass slide. IHC immunostaining for IL-13Rα2 was performed as described previously (Joshi et al. (2008) Neuro Oncol, 10:265-274). Briefly, IL-13Rα2 expression in PDAC and normal tissues were determined by using goat polyclonal antibody against IL-13Rα2 (R&D, Minneapolis, MN). The sections were deparaffinized, dehydrated with a gradient of alcohol from 100%, 75% and 50% and treated with antigen unmasking reagent to unmask the IL-13Rα2 protein. Autofluorescence in paraffin tissue sections was minimized by incubating with 1% Sodium borohydride solution for 2 hours and incubated in block buffer consisting of 5% rabbit serum and 1% biotin free bovine serum albumin in PBS for 2 hours. The paraffin tissue sections were immunostained with IL-13Rα2 antibody (0.5 μg/ml) overnight at 4° C., washed twice with 1×PBS and incubated with biotinylated rabbit anti-goat antibody (0.5 μg/ml). The sections were then reacted with Streptavidin Alexa Fluor® 594 secondary antibody (0.5 μg/ml) for 45 minutes, washed twice with 1×PBS and incubated with biotinylated-anti-streptavidin antibody (1 μg/ml) for 45 minutes to amplify the fluorescent signal. In the final step, the samples were incubated with Streptavidin Alexa Fluor® 594 secondary antibody (0.5 μg/ml) for 45 minutes at 22° C. (room temperature). After three washes with 1×PBS, the sections were mounted and cured with Vectashield® antifade mounting medium (Vector Laboratories, Burlingame, CA) and viewed in a fluorescence microscope using Rhodamine filters (Chroma, Rockingham, VT). The samples were immunostained with isotype control goat IgG in parallel, which served as negative controls.

The PDAC tumor sections were evaluated and graded for IL-13Rα2 expression by independent investigators at different time points in a blinded fashion. A positive field is defined as a number of immunostained positive area representing a cluster of more than 50 positive cells counted at 200× magnification. The summation of % positive area immunostained in the tissues sections was counted at this magnification by individual investigator in a blinded manner. The extent of immunostaining was also documented on a semi-quantitative scale (<1+, 1+, 2+ and 3+). The findings were decoded after staining and counting % positive fields, and data were analyzed. Immunostaining score values of 0 and 1+ were considered negative for IL-13Rα2 expression (negative tumors), while 2+ and 3+ were considered as moderately positive and strongly positive, respectively (positive tumor). IHC of PDAC samples were evaluated by at least a team of total six investigators, which consisted of anatomical, clinical and research pathologists and experienced specialists who were blinded to the clinical data. Final evaluations of ambiguous observations were decided after discussing with all investigators.

### In Situ Hybridization Analysis (ISH)

IL-13Rα2 mRNA in PDAC and normal pancreas samples was evaluated by using Qdot® 525 labeled anti-sense riboprobe (Thermo Fisher Scientific). The specimens were deparaffinized, dehydrated, and treated with 25 mM Sodium-citrate buffer, pH 6.5 for 20 minutes for antigen retrieval as described above. The samples were washed with 1×PBS and incubated with 5 μg/ml proteinase K (Sigma-Aldrich, St. Louis, MO) for 15 minutes for permeabilization and then DNase (5 units/ml) to destroy residual DNA in the tissue section by incubating for 6 hours at room temperature. The tissue sections were washed with 1×PBS and hybridized with an in vitro transcribed biotinylated antisense riboprobe for detection of IL-13Rα2 RNA after dissolving in 2× hybridization buffer (4×SSC, 0.2M Sodium phosphate (pH6.5), 2×Denhardt's solution, 0.1 mg/ml Sodium azide) and 20% Dextran Sulfate solution. An in vitro transcribed biotinylated sense riboprobe for IL-13Rα2 was included as a negative control. The PDAC tissue sections were then incubated with 0.5 μg/ml streptavidin-Qdot® 525 for 45 minutes, washed three times with 1×PBS, incubated with biotinylated anti-streptavidin antibody and streptavidin-Qdot® 525 (0.5 μg/ml) for 45 minutes for amplification of the hybridized signals. The slides were washed three times with 1×PBS, dried and mounted with Vectashield® antifade mounting medium and viewed under a fluorescence microscope using Qdot® 525 filters. The fluorescence microscopic images were acquired, digitized and analyzed using Nikon-S-Elements software (Nikon Instruments Inc., Melville, NY). The tissue sections were evaluated and graded for IL-13Rα2 mRNA hybridized fluorescence intensity by two investigators independently at different time points in a blinded fashion.

### Statistical Analysis

IHC and ISH analysis of IL-13Rα2 for PDAC tissue specimen precision study was completed using twelve readings (replicates) by six investigators in which each slide was evaluated at two independent and separate time-points for clinical stage and pathologic grade. Analysis of these precision data for the six replicate sets was completed by using exact binomial proportion with exact two-sided P-values. For the trend analysis to assess any change in IL-13Rα2 over grade or stage of the subjects, Cochran-Armitage statistics (Agresti et al. (2002) Biostatistics, 3:379-386) was calculated using exact two-sided P-values. Exact inference is a nonparametric technique, which does not require any distributional expectations about the population of interest (Corcoran et al. (2002) Journal of Modern Applied Statistical Methods, 1:42-51). Multiple (or multivariable) logistic regression (Hosmer et al. (2000) Applied logistic regression, 2nd ed.; John Wiley and Sons, Inc.: New York, Chichester, Weinheim, Brisbane, Singapore, Toronto) was also done by adjusting for age and gender in the stage database and age in the Grade database. All statistical analyses in the present study were performed using SAS Software 9.3 (SAS Institute Inc., Cary, NC). The overall survival curves were prepared according to the Kaplan-Meier method and the differences were analyzed by the Log-rank test. A multivariate analysis using all clinicopathological parameters and prognostic factors was performed using the Cox's proportional hazards, where the results were scored as hazard ratio (HR), 95% confidence interval (CI), and P-value (P<0.05 indicated significance). A second multivariate analysis was performed using the parameters identified as significant through a univariate analysis of the individual clinical sites, NTT and YCU, for comparison. Correlation between the variates was evaluated using Pearson's (R), point-biserial (R_{bis}), and phi (φ) correlation coefficients (GraphPad PRISM 8.1 Software, Inc., La Jolla, CA), and all statistical analyses were performed using PASW Statistics version 22 (IBM Corporation, Armonk, NY).

### Cloning IL-13BAD

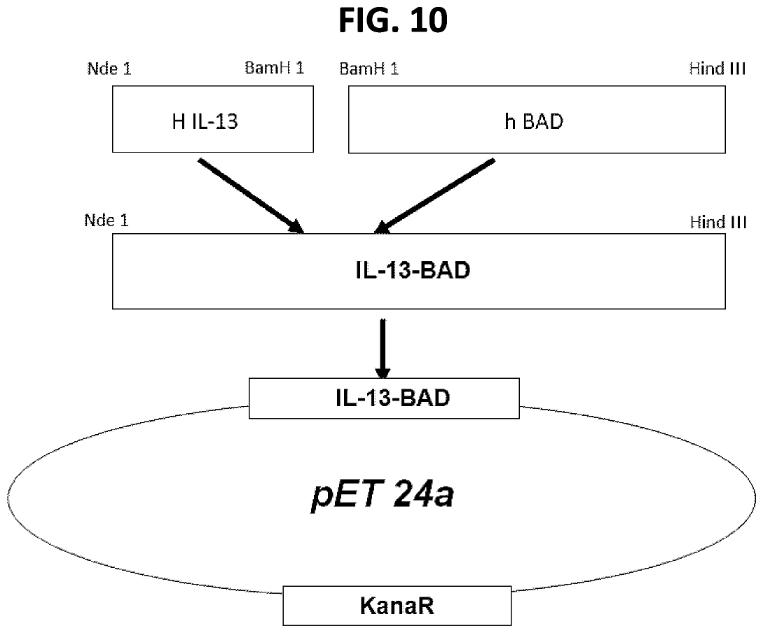

Mature human interleukin-13 was cloned from total RNA isolated from human peripheral blood mononuclear cells (PBMCs), which were stimulated with 10 μg/ml of lipopolysaccharide for 24 h. Total RNA was extracted from the PBMCs using the RNeasy RNA extraction kit (Qiagen, Valencia, CA) and reversed transcribed with Moloney murine leukemia virus reverse transcriptase. The primers amplify the amino acids serine (19) to asparagine (133) that include matured form of human IL-13 (pMPL13) with NdeI restriction site (in bold) at 5′-TAATTTGCCCATATGTCCCCAGGCCCT (forward primer; SEQ ID NO: 13) and BamH1 site (in bold) at 3′-GAAGTTGGATCCTGTTGAACCGTCCCTCGC (backward primer; SEQ ID NO: 14). The chimeric construct was generated by ligating the NdeI and BamH1 digested PCR product with NdeI and BamH1 restriction digests of GM-CSF-BADaa plasmid DNA to generate IL-13-BAD aa (IL-13-BAD) with NdeI at 5′ and HindII at 3′in pET expression vector (see, Antignani and Youle, Biochemistry, 15; 44(10):4074-82, 2005). The vector configuration for this construct is shown in FIG. 10.

### Cloning IL-13-FADD

Similarly, mature human interleukin-13 clone derived from total RNA isolated from human peripheral blood mononuclear cells (PBMCs) was used to produce a chimeric construct IL-13-FADD. Briefly, the IL-13 clone with NdeI at 5′ and BamH1 site at 3′ was used for constructing IL-13-FADD chimeric construct as described above. The coding region of human FADD (gene accession number (NCBI, NM_003824.4) was modified at 5′ end for constructing BamH1 site and Hind III at 3′ site. The primers amplify the amino acids are shown below:

(SEQ ID NO: 15)
FW-FADD: GTTAAAGGATCCGACCCGTTTCTGCTC
(SEQ ID NO: 16)
BW-FADD: TCCTGTAAGCTTTCAGGACGCTTC

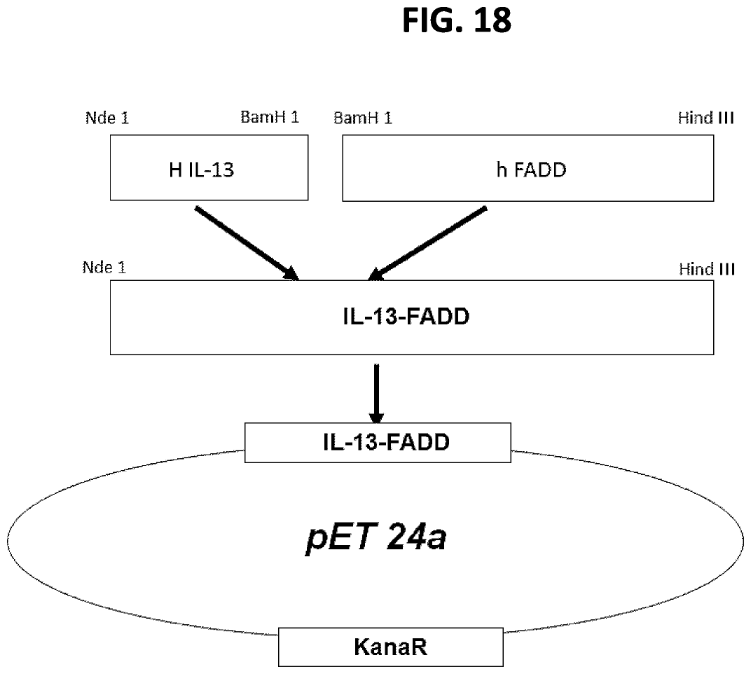

The chimeric construct for IL-13-BAD was generated by ligating the NdeI and BamH1 digested PCR product with either NdeI and BamH1 restriction digests of IL-13 and GM-CSF-BAD plasmid DNA to generate IL-13-BAD. FADD (accession #NM_003824) was derived from PCR product after constructing BamH1 at 5′ and Hind III at 3′ sites using gene specific primers as shown above to develop IL-13-FADD construct. The vector configuration for this construct is shown in FIG. 18. The chimeric constructs were then cleaved with NdeI and Hind III restriction enzymes and ligated with previously digested pET24a vector to generate IL-13-BAD and IL-13-FADD in pET24a prokaryotic expression system. The vector and insert sequences at the junctions were confirmed by performing ABI PRISM® sequence reactions.

Optimization of Protein Expression in E. coli

Bacterial transformation for expression of IL-13-BAD was performed by incubating BL21(DE3)pLysS bacterial strains (Invitrogen, WI) with IL-13-BAD plasmid DNA on ice for 30 minutes and heat/shock treatment for 30 seconds as per the manufacturer's instruction. The transformed bacteria were added to 250 μl of SOC enriched medium and shaken for 2 hours at 37° C. in a bacterial shaker. 75 μl of transformed bacteria were plated on a LB plate with kanamycin plus chloramphenicol (35 μg/ml) in case of BL21(DE3)pLysS cells and incubated overnight in an air incubator for colony formation. Each single colony on the LB plate was inoculated into 5 ml of LB broth containing 15 μg/ml kanamycin and 35 μg/ml chloramphenicol and incubated overnight at 37° C. in a bacterial shaker. 100 μl fresh transformed bacterial preparation was added to 100 ml of enriched superbroth (Biowhittakar, Walkersville, MD) that contained 15 μg/ml kanamycin, 20 ml of 20% glucose and 12 ml of 4% magnesium sulfate. The bacteria were induced with 0.25 to 2.5 mM of IPTG when the optical density of the bacteria reached between 0.5 and 0.6 OD and shaken for an additional 1 to 8 hours in a bacterial shaker at 37° C. One ml of the bacteria was taken out at 1, 2, 3, 4,5, 6 and 8 hr, centrifuged and lysed. 5 μl of cell lysate was electrophoresed on a 10% SDS-PAGE for evaluation of protein expression.

### Denaturation, Refolding and Purification of the Cytotoxin

The bacterial pellet from the one-liter culture was washed with 50 mM Tris-HCl PH 7.4, 1 mM sodium EDTA and 20% sucrose. The BL21(DE3) pellet was washed with 70 ml cold water and incubated on ice for 20 minutes with intermittent shaking. The bacteria were centrifuged at 8000×g for 30 minutes to separate out the periplasm and sphereoblasts. This step was not needed for the BL21(DE3)pLysS pellet as lysozyme molecules were expressed during induction and released upon freeze-thawing of the pellet. At the end of 1 hr incubation, 4 ml of 5M NaCl was added to the suspension followed by 10 ml of 25% Triton™-X-100 and mixed well after each addition. The bacterial suspension was further incubated for one hour at room temperature. The inclusion bodies released from spheroblasts were washed five times with 50 mM Tris-HCL PH 7.4 containing 20 mM EDTA (50/20 mM Tris buffer). A parallel spheroblast pellet obtained from an equal amount of bacteria was washed three times with 50/20 mM Tris buffer with 0.1% Triton-X 100 and two additional washings with 50/20 mM Tris buffer without Triton-X 100. After washing, the inclusion bodies were denatured with 10 ml of 7M guanidinium-hydrochloride dissolved in 50 mM Tris-HCl pH 8.0 and 65 mM dithiothreitol (DTE) for 4, 6, 8, 16 and 24 hours.

Refolding of protein from the solubilized inclusion bodies was optimized by refolding 5, 10, 15, or 20 mg protein per ml diluted to 33.3 μg-133.3 μg/ml in refolding buffer. Refolding buffer that contained 0.6M arginine and 0.9 mM oxidized glutathione was used for refolding of denatured protein at 10° C. for 48 hours. The refolded preparation was dialyzed against 10 mM Tris-HCl pH 7.4 buffer containing 60 mM urea. The chimeric protein was purified by Fast Protein Liquid Chromatography using Q Sepharose®, Mono Q® and Sephacryl® S-100 gel exclusion columns (General Electric Health Sciences). The purified protein was electrophoresed on a10% SDS-PAGE gel and stained with Coomassie Blue. The gel was de-stained with 7% acetic acid and 5% methanol (v/v). IL-13-BAD appeared to be induced equally well with IPTG and purified to single band entities demonstrating high purity of the protein products. The chimeric proteins migrated at approximately 40 kDa as expected.

### Cytotoxic Activity of IL-13-BAD

Cell lines U251, A172, T98G, HS766T HPAF-III, HN12 and UCUM911 were purchased from the American Type Culture Collection® (ATCC®) or DCTD, NCI or gift from YCU, Tokyo; Japan and maintained in recommended culture medium to access the cytotoxicity of these recombinant proteins. Briefly, cells in 100 μL were incubated at concentrations of 10×10⁴ cells/mL in 96-well microtiter plates overnight and treated with various concentrations of IL-13-BAD protein for 96 h in RPMI 1640. The results were expressed as a percentage of control cells. Cell viability was determined with the trypan blue exclusion technique. Values represent the mean of triplicate samples with a <10% standard error of the mean.

### Colony Formation Assay

In vitro cytotoxic activity of IL-13-BAD on human glioma, pancreatic and SCCHN cell lines was evaluated by a colony formation assay. Cells were harvested from culture, washed and resuspended in complete medium. Cells were plated in quadruplicate in 10-cm2 Petri dishes (Falcon; Becton Dickinson, Lakeridge, NJ) and cultured overnight. The number of cells per plate was selected so that more than 100 colonies were obtained in the control group. The cultures were then incubated with IL-13-BAD (0-1000 ng/ml) for 8 days at 37° C. in a humidified CO₂ incubator. After removing the medium, the colonies were washed with PBS and stained with 0.025% crystal violet in 25% ethyl alcohol. Colonies with 50 or more cells were scored. The number of colonies observed in IL-13-BAD treated cultures was expressed as a percentage of the number of colonies formed in untreated control cultures.

### Example 2

### Patient Demographics and Clinicopathological Analysis

Two PDAC sample sets derived from two different institutions were combined and independently evaluated for IL-13Rα2 expression and correlated with clinical data. The demographic information of patients from the two hospitals are shown in Table 1.

TABLE 1
Demography of PDAC patients.
Sex

| | | |
|---|---|---|
| Male | 148 | (63) |
| Female | 88 | (37) |

| | |
|---|---|
| Median age | 65.3 ± 5 |

Tumor Location

| | | |
|---|---|---|
| Head | 161 | (68) |
| Body & Tail | 75 | (32) |

Clinical Stage (UICC)

| | | |
|---|---|---|
| I | 28 | (12) |
| II | 89 | (38) |
| III | 85 | (36) |
| IV | 34 | (14) |

Pathologic Findings

| | | |
|---|---|---|
| Well differentiated | 80 | (34.5) |
| Moderately differentiated | 131 | (55.5) |
| Poorly differentiated | 25 | (10.5) |
| Diabetes (before surgery) | 77 | (33) |

| | |
|---|---|
| CA 19-9 (U/mL) | 1037 ± 202 |

Eighty of the 236 patients (34.3%) had well differentiated, 55.5% moderately differentiated and 10.2% poorly differentiated disease. Among these, 12% patients presented with clinical stage I, 38% with stage II, 36% with stage III and 14% with stage IV disease. There were 63% male and 37% female patients. These patients had different clinicopathological presentations of cancer invasion and survived 9-17 months with poorly differentiated grades and 18-29 months with well-to-moderately differentiated grades. Clinicopathological Analysis and patient survival is shown in Table 2. The patients with invasive disease to PL and Ne survived relatively shorter than those with well or moderately differentiated disease.

### Example 3

IL-13Rα2 Expression Correlates with PDAC Grade and Clinical Stage

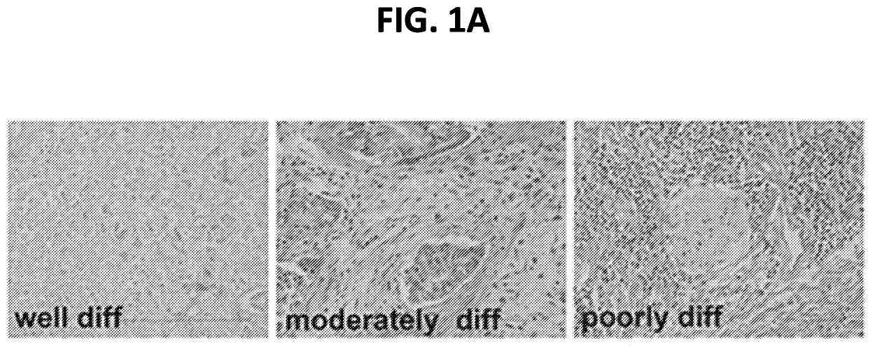

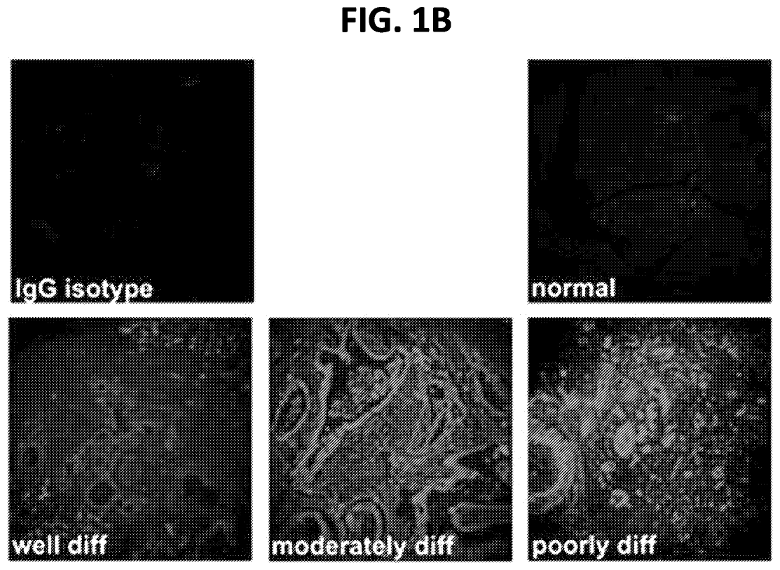

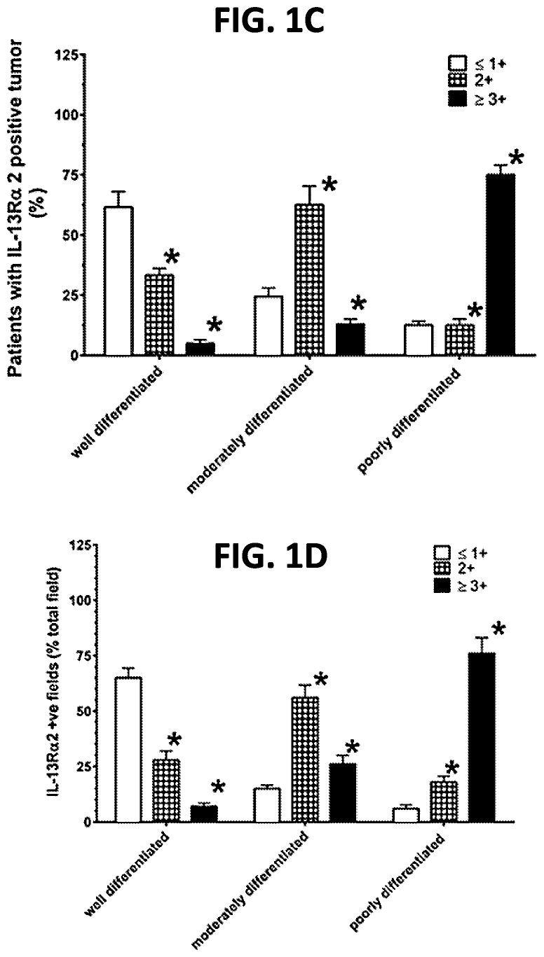

The expression of IL-13Rα2 in 236 PDAC samples with different pathological grades and clinical stages was examined. Typical H&E and immunofluorescence immunostaining patterns for IL-13Rα2 expression in pancreatic cancer samples is shown in FIGS. 1A and 1B. Further, IL-13Rα2 subunit expression was examined in 236 PDAC samples with different pathological grade and clinical stage database. The intensity of IL-13Rα2 subunit expression by IHC was analyzed by six investigators in a blinded fashion. To assess precision of the pathological Grade database, 236 PDAC samples were evaluated by six investigators on two different occasions resulting in twelve different sets of readings to score IL-13Rα2 immunostaining intensity. Similarly, 236 PDAC samples in the clinical stage database were also examined, which generated twelve independent readings for each slide. Immunostaining intensity scores of ≥2+ were considered positive, while ≤1+ were considered negative. A very high concordance among these twelve sets of readings was observed in both databases, where scores 3+ and 4+ were combined as 3+, while scores 1+ and no score (≤1+) were combined as 1+. 214/236 (90.6%) samples showed concordant scores (exact 95% CI=76.8-96.6) for pathological Grade database and 202/230 (87.8%) samples demonstrated concordant scores of (exact 95% CI=71.2-92.1) for clinical stage database. Our results exhibited high precision in the IHC for both sets of databases. For the remainder of the data analysis, the discordant score values were determined by assigning the majority score and when there was an equal number of discordant scores, the lower score was considered. For the rest of the analysis, all discordant cases were considered as negative for IL-13Rα2 expression. The final score is the median±SD of these twelve sets. These results showed high precision for both databases.

As shown in FIG. 1, IHC results show that the extent of immunostaining for IL-13Rα2 is significantly higher as the grade advances from well differentiated to moderately to poorly differentiated PDAC tumor samples (FIGS. 1A and 1B). The combined positive values of IL-13Rα2 (2+ and 3+) immunostaining intensity showed a statistically significant higher trend with the pathological grade of the disease (well differentiated=38.3%, moderately differentiated=75.6%, poorly differentiated=87.5%; Exact P<0.0001) in comparison with negative values of IL-13Rα2 (≤1+) (FIG. 1C). Similarly, a significantly increasing trend was also detected even after adjusting for age (P=0.0025) in this cohort (data not shown). In contrast, 12 normal pancreas specimens showed ≤1+ staining for IL-13Rα2 (FIG. 1B). Similarly, the percentage of positive fields for IL-13Rα2 expression in 2+ and 3+ positive specimens in IHC analysis demonstrated highly significant trend in these specimens (well differentiated=35.0%, moderately differentiated=82.0% and poorly differentiated=94%; Exact P<0.0001) compared to PDAC samples with negative values of IL-13Rα2 (≤1+) (FIG. 1D). It was observed that only tumor cells showed appreciable IL-13Rα2 immunostaining. It is possible that IL-13R a2 is also expressed in stroma and immune cells, however, below the detection limits of the IHC and ISH techniques used here. A majority of the tumor cells have membranous and cytoplasmic immunostaining.

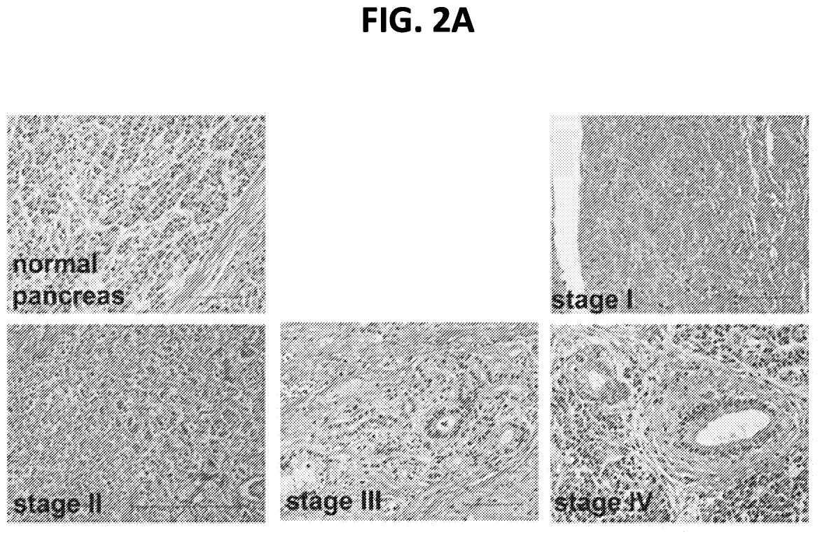

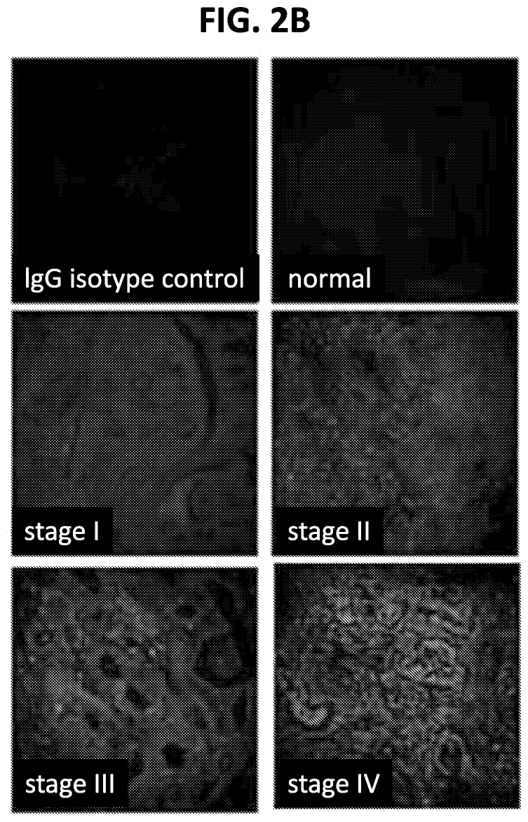

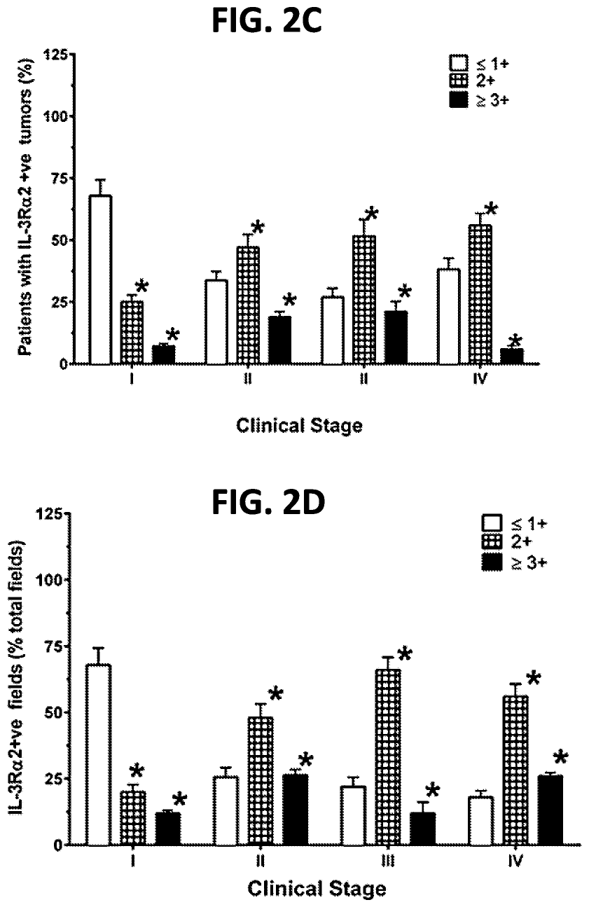

PDAC samples with different clinical stages revealed a significant increase in the proportion of patients as the stage advanced from stage I to III-IV for intensity of immunostaining of IL-13Rα2 expression (FIGS. 2A and 2B). Interestingly, the combined IHC positive values for IL-13Rα2 (≥2+ and 3+ combined) in comparison to samples with negative values (≤1+) showed a statistically significant increasing trend with the clinical stages (stage I=32.1%, stage II=66.3.2%, stages III-IV=69.7%; Exact P≤0.0001). This pattern of IL-13Rα2 expression was also observed even after adjusting for age and gender (P=0.002, data not shown). Twelve normal pancreas specimens showed ≤1+ staining for IL-13Rα2 (FIG. 2B). Similarly, the percentage of positive fields for IL-13Rα2 expression in 2+ and 3+ positive specimens in IHC analysis demonstrated highly significant trend in these specimens with different stage of disease (stage I=21.4%, stage II=47.2% and stage III=84.9%; Exact P<0.0001) compared to negative values of IL-13Rα2 (≤1+) (FIG. 2D).

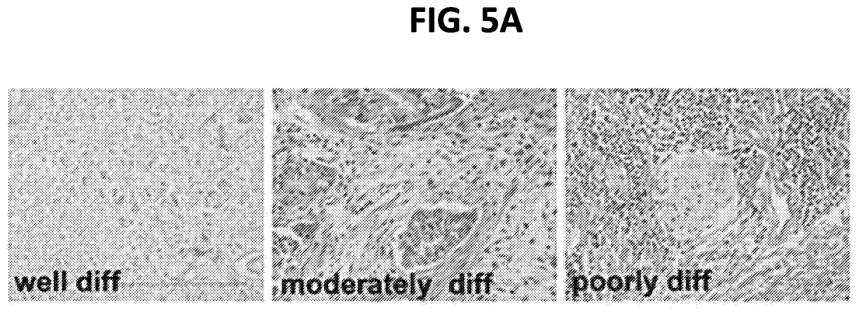

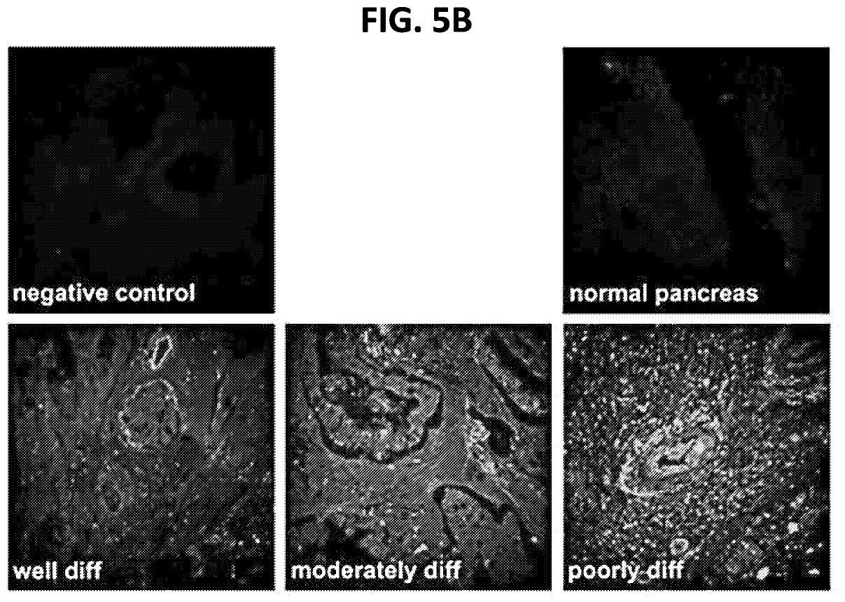

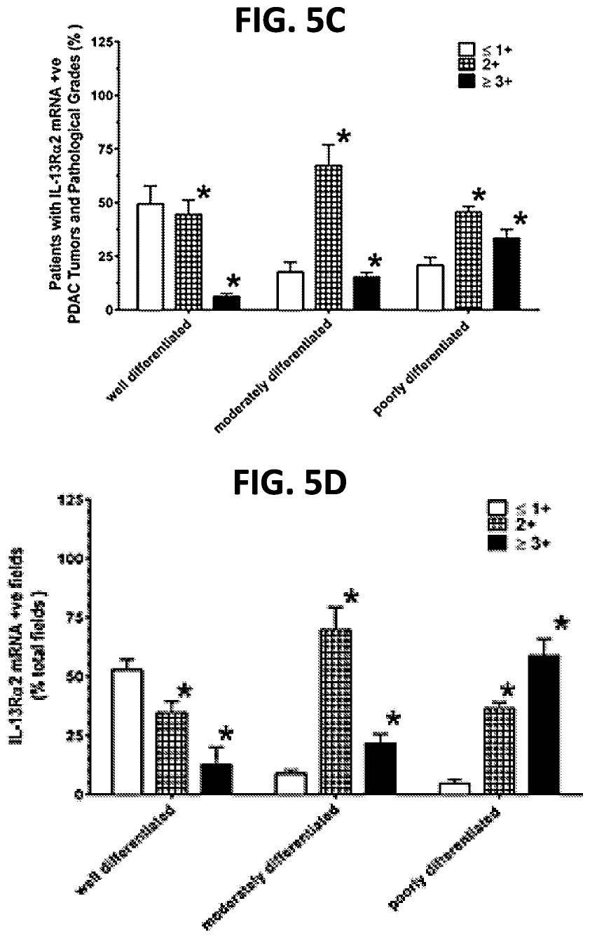

As shown in FIG. 5A-5D, ISH data reveal that the levels of IL-13Rα2 mRNA also showed a significant increase in hybridization intensity for IL-13Rα2 mRNA (FIG. 5B) with the well differentiated to moderately to poorly differentiated grades (H&E images shown in FIG. 5A) corroborating the IHC analysis. The total number of samples with positive hybridization for IL-13Rα2 (2+ and 3+) also demonstrated a statistically significant increasing pattern with the pathologic grade (well differentiated=50.6%, moderately differentiated=82.4%, poorly differentiated=79.2%; Exact P<0.00014) compared to PDAC tumors with negative values of IL-13Rα2 (≤1+) (FIG. 5C). The significant increasing trend in PDAC samples was also observed even after adjusting for age (P<0.0022) in this cohort (data not shown). In contrast, 12 normal pancreas specimens showed≤1+ intensity for hybridization for IL-13Rα2 mRNA. Similarly, the percentage of positive fields for IL-13Rα2 mRNA in 2+ and 3+ positive specimens in ISH analysis demonstrated highly significant trend in these specimens (well differentiated=47.2%, moderately differentiated=91.2% and poorly differentiated=95.4%; Exact P<0.00011) compared to samples with negative values of IL-13Rα2 (≤1+) (FIG. 5D).

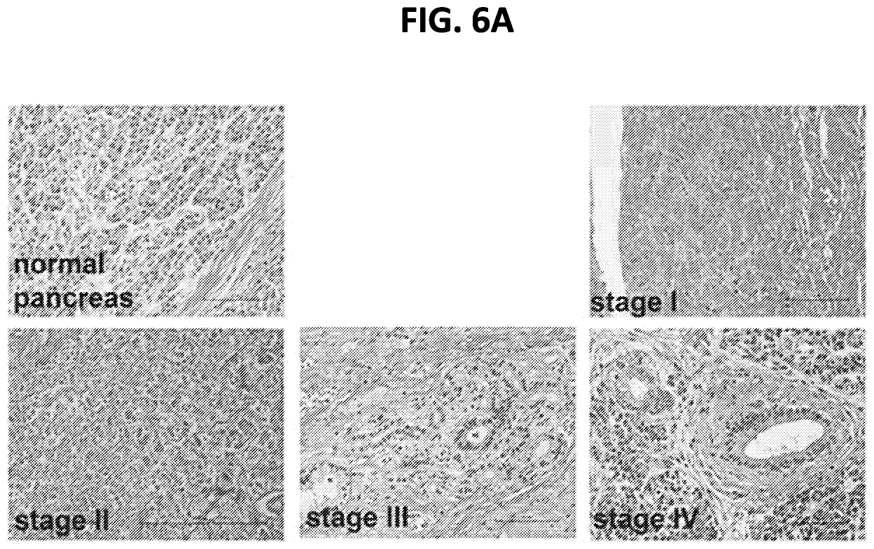

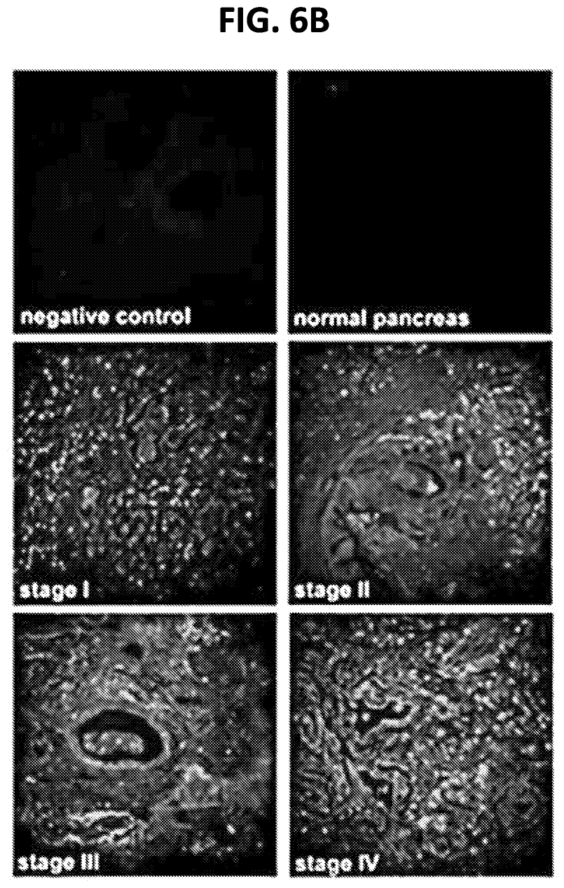

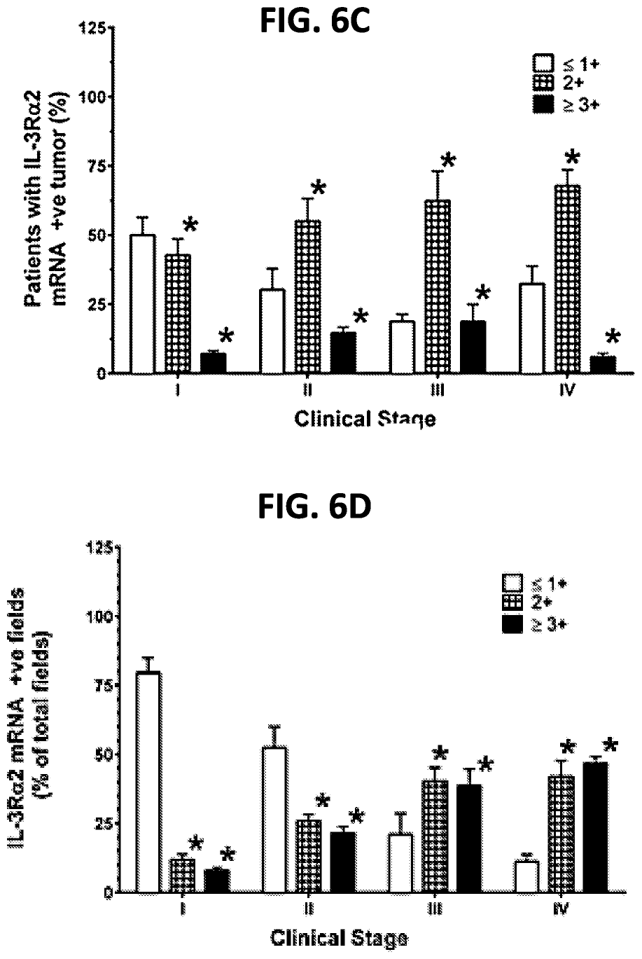

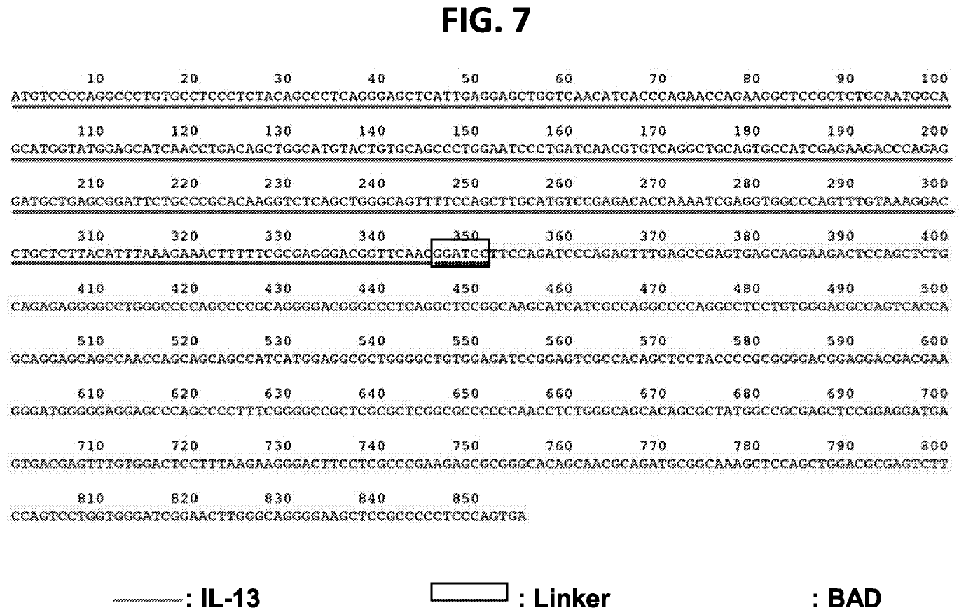

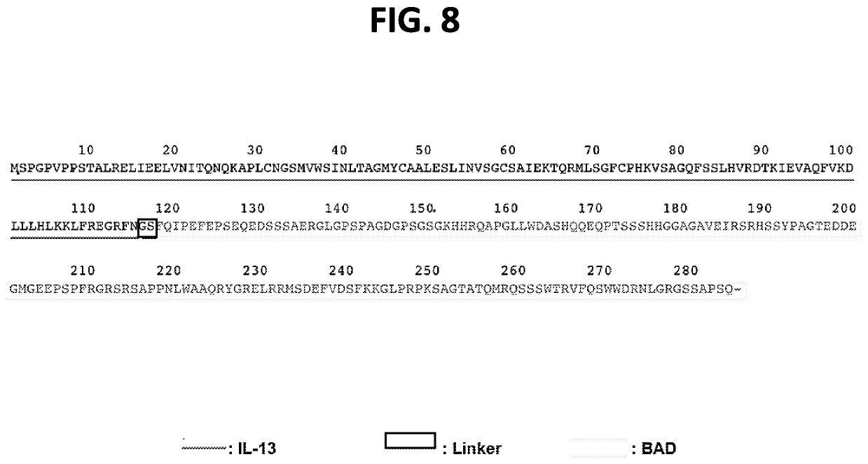

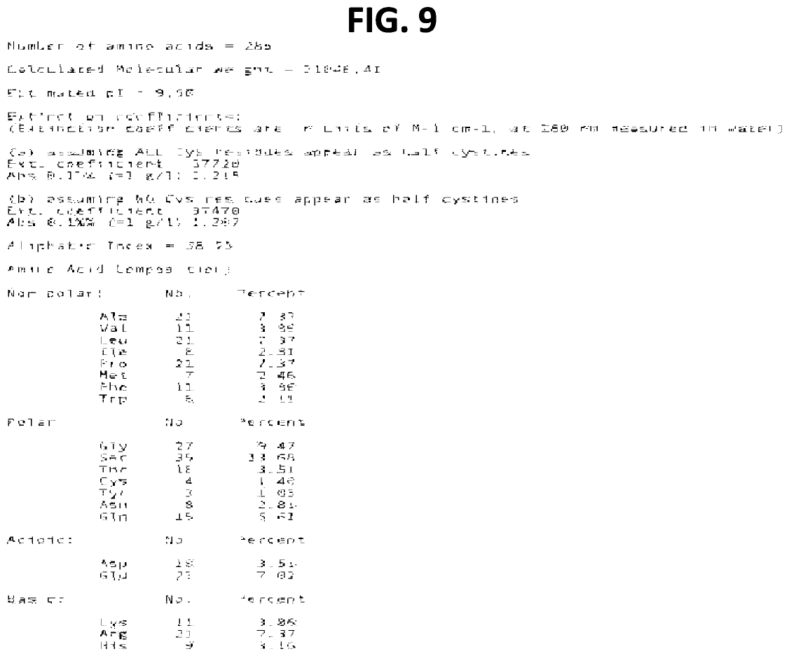

PDAC samples with different clinical stages confirmed a significant increase in IL-13Rα2 mRNA in samples as the stages progressed from stages I to III-IV for ISH intensity (FIG. 6B). H&E staining of these samples is shown in FIG. 6A. Interestingly, the intensity of hybridization for positive values of IL-13Rα2 (2+ and 3+ combined) when compared to samples with negative values of IL-13Rα2 (≤1+) showed a statistically significant increasing pattern with the stage of disease (stage I=50.0%, stage II=69.7%, stages III-IV=77.3%; Exact P≤0.0001) (FIG. 6C). This trend was continued even after adjusting for age and gender (P=0.002, data not shown). Similarly, the percentage of positive fields for IL-13Rα2 mRNA expression in 2+ and 3+ positive specimens in ISH analysis demonstrated highly significant trend in these specimens (stage I=21.4%, stage II=47.2%, stages III-IV=84.9%; Exact P<0.00011) compared to samples with negative values of IL-13Rα2 (≤1+) (FIG. 6D).

### Example 4

Heterogeneity in IL-13Rα2 Expression is Associated with Clinicopathological Attributes of PDAC

A total of 69 (29.5%) and 160 (67.7%) of 236 PDAC specimens with moderately to poorly differentiated pathological grade, respectively, were detected with invasion of the peripancreatic neuroplexus (PL) and nerve in the pancreas (Ne) as evident by H&E staining. The remainder of the samples from the PL and Ne groups, 167/236 (70.5) and 76/236 (32.3%) were identified as well differentiated pathological grade samples (see Table 2).

Data was stratified based on IHC immunostaining for IL-13Rα2 positivity, which revealed that 56 (36%) samples demonstrated IL-13Rα2 positive tumor cells that had invaded PL were of moderate to poor pathological grade. It was observed that 100 (64%) specimens with IL-13Rα2 positive immunostaining did not show PL invasion and were of well differentiated pathologic grade. Interestingly, 13 (16%) PL invasion positive samples with moderately to poor grade also revealed IL-13Rα2 positive immunostaining. The remaining 67 (84%) samples with no invasion of the PL and no IL-13Rα2 positive immunostaining were well differentiated.

In contrast, the number for IL-13Rα2 positive PDAC specimens rose to 117 (75%) with Ne invasion while 39 (25%) samples with no Ne-invasion were positive for IL-13Rα2 (P=≤0.01; Table 3). The number of IL-13Rα2 negative and Ne positive PDAC was 43(54%) while 37(46%) were negative for both IL-13Rα2 and Ne. Interestingly, the samples with no invasion of Ne were well differentiated which is similar to the samples with no invasion to PL. Excluding patient survival, IL-13Rα2 expression strongly (correlation index>0.2) and significantly (P-value<0.05) correlated with invasion to peripancreatic neuroplexus (PL), to back constructs of pancreas (RP), and nerve in the pancreas (Ne). These two parameters that were invasion to peripancreatic neuroplexus (PL) and nerve (Ne), were correlated with IL-13Rα2 expression in terms of patient survival.

TABLE 2
Clinicopathological Analysis and patient survival.

| | | Number | Median | |
|---|---|---|---|---|
| | Compar- | (of pa- | Survival | P- |
| Pathological Findings | ison | tients) | (months) | value |
| Differentiation | Well:mod- | 081:155 | 21:17 | 0.0005 |
| | poor | | | |

Invasion

| | | | | |
|---|---|---|---|---|
| . . . to bile duct (CH) | −ve:+ve | 133:103 | 20:16 | 0.027 |
| . . . to duodenum (DU) | −ve:+ve | 163:073 | 21:13 | 0.005 |
| . . . to front constructs of | −ve:+ve | 127:109 | 22:14 | 0.001 |
| pancreas (S) | −ve:+ve | 078:158 | 29:15 | ≤0.001 |
| . . . to back constructs of | | | | |
| pancreas (RP) | | | | |
| . . . to portal vein (PV) | −ve:+ve | 172:064 | 22:11 | ≤0.001 |
| . . . to artery (A) | −ve:+ve | 222:014 | 19:09 | 0.003 |
| . . . to peripancreatic | −ve:+ve | 167:069 | 23:11 | ≤0.001 |
| neuroplexus (PL) | | | | |
| . . . to the other | −ve:+ve | 223:013 | 18:12 | 0.011 |
| organs (OO) | | | | |
| . . . to lymph duct in the | ≤1:≥1 | 162:074 | 22:12 | ≤0.001 |
| pancreas (Ly) | | | | |
| . . . to vein in the | ≤1:≥1 | 095:141 | 27:14 | 0.002 |
| pancreas (V) | | | | |
| . . . to nerve in the pancreas | ≤1:≥1 | 076:160 | 29:15 | ≤0.001 |
| (Ne) | | | | |
| . . . to main pancreatic duct | −ve:+ve | 124:112 | 21:16 | 0.317 |
| (Mpd) | | | | |

Pathological findings including invasion to different parts of pancreas were analyzed to determine MST and grades of PDAC. P ≤ 0.05 were considered significant.

### Example 5

Association of IL-13Rα2 with Perineural Invasion

The association of IL-13Rα2 expression with perineural invasion of the pancreatic cancer was analyzed. IL-13Rα2-positive cancers showed 36% (56/156) invasion of peripancreatic neuroplexus (PL) while IL-13Rα2-negative cancer showed only 16% (13/80) in the combined data from both hospitals (Table 3). Similarly, IL-13Rα2-positive cancer samples showed 75% (117/156) Ne, but IL-13Rα2-negative cancer showed only 54% (54/80). Number of PL and Ne positive PDAC were found significantly higher in the IL-13Rα2-positive cancers. Interestingly, a greater number of tumor samples with invasion to Ne rather than PL were significantly associated with enhanced IL-13Rα2 detected by IHC and mRNA (ISH) (P≤0.0001) in PDAC patients with moderately to poor pathologic grade.

TABLE 3
Analysis of IL-13Rα2 in Perineural Invasion in PDAC.

| | | Patients with IHC staining for IL-13Rα2 | | | | |
|---|---|---|---|---|---|---|
| | | Positive | | MST | Negative | MST |
| Pathologic type | | (≥1+) | | (months) | (≤1+) | (months) |
| PL | Positive | 56 | (36%) * | 10 | 13 (16%) | 27 |
| | Negative | 100 | (64%) | 18 | 67 (84%) | 34 |
| Ne | Positive | 117 | (75%) * | 13 | 43 (54%) | 27 |
| | Negative | 39 | (25%) | 17 | 37 (46%) | 29 |

PL = Invasion to peripancreatic neuroplexus; Ne = Invasion to nerve in the pancreas; IL-13Rα2 and MST were compared in patients with invasion in PL and Ne.
* P = ≤0.0001.

### Example 6

IL-13Rα2 is Associated with Overall Survival of PDAC Patients

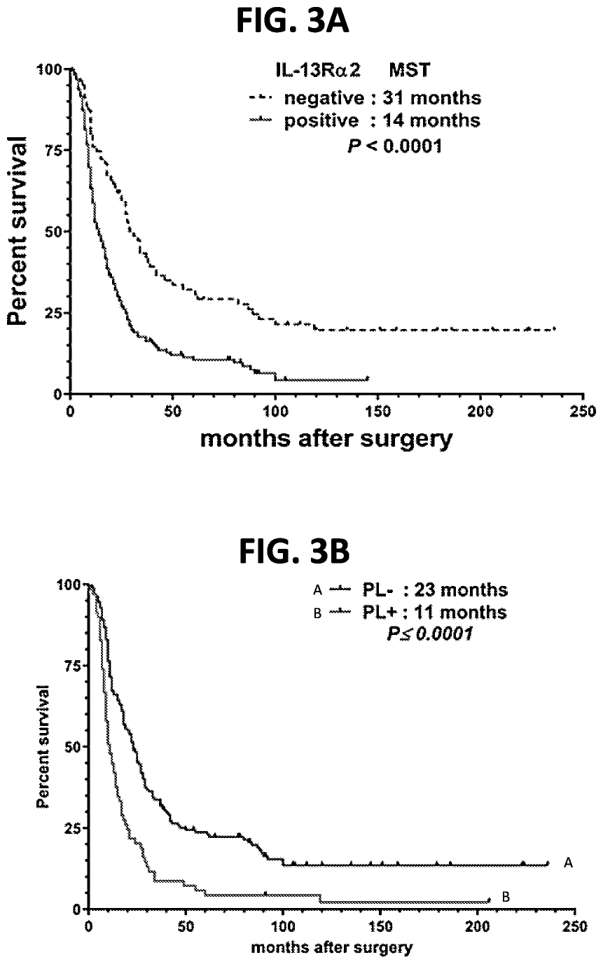

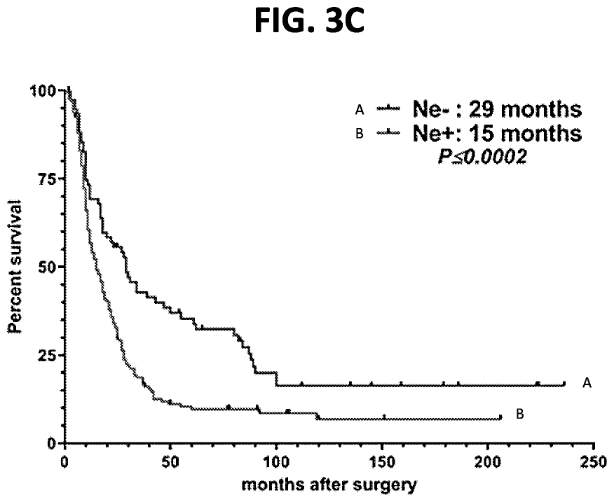

A Kaplan-Meier survival analysis was performed for all 236 PDAC patients, which revealed that the median survival time (MST) of 80 patients with IL-13Rα2-negative tumors were 31 months compared to 14 months in the 156 IL-13Rα2-positive PDAC patients (FIG. 3A). Log-Rank test analysis revealed that survival time of patients with IL-13Rα2-positive tumors was significantly shorter than patients with negative tumors in the combined data from NTT and YCU hospitals (HR; 2.214, 95% CI; 1.475-2.954, P<0.0001). The results were sub-stratified for patients with PL−/PL+ and Ne−/Ne+ diagnosis. As shown in FIG. 3B, the MST for PL− patients was 23 months compared to 11 months for PL+ patients (P≤0.0001); and 29 months for Ne− and 15 months for Ne+ patients (P≤0.0002; FIG. 3C).

### Example 7

### Analysis of Clinicopathological Findings and Prognostic Factors

The clinicopathological parameters were investigated using Log-rank test for relationship to patient survival in the combined dataset (Table 2). Fifteen parameters including tumor size, differentiation, invasion of bile duct (CH), to duodenum (DU), to front constructs of pancreas (S), to back constructs of pancreas (RP), to portal vein (PV), to artery (A), to peripancreatic neuroplexus (PL), to the other organs (OO), to lymph duct in the pancreas (Ly), to vein in the pancreas (V), to nerve in the pancreas (Ne), UICC-stage, and CA19-9, significantly affected patient survival on univariate analysis. Sixteen parameters that included IL-13Rα2 expression and 15 clinicopathological parameters were also analyzed by multivariate analysis by Cox's proportional hazards model. Only 4 parameters including IL-13Rα2 expression, UICC-clinical stage, CA19-9, and invasion to front constructs of pancreas (S) were independent prognostic factors on multivariate analysis (Table 2). In addition, an extensive analysis was performed to determine any correlation between adjuvant chemotherapy and IL-13Rα2 expression or prognosis of these patients, however, no correlation between IL-13Rα2 expression and adjuvant therapy was observed. Furthermore, none of the subjects included in this study had received neoadjuvant chemotherapy (data not shown).

Ten parameters significantly affected patient survival at NTT hospital while 14 parameters affected patient survival at YCU hospital by univariate analysis. Multivariate analysis revealed that only 4 parameters including IL-13Rα2 expression, UICC-stage, tumor differentiation, and invasion of lymph duct (Ly), and 3 parameters that included IL-13Rα2 expression, UICC-stage, and CA19-9 were independent prognostic factors (Table 1). IL-13Rα2 expression and UICC-stages were found to be common prognostic factors in the combined data from the two hospitals by multivariate analysis. The multivariate analysis was performed using Cox proportional hazard model with all the clinicopathological parameters and prognostic factors. A second multivariate analysis was performed for comparison using solely the parameters that were observed to be significant by univariate analysis of the data from each clinical site, NTT and YCU.

### Example 8

IL-13Rα2 in PNI is Correlated with Decreased Survival in PDAC Patients

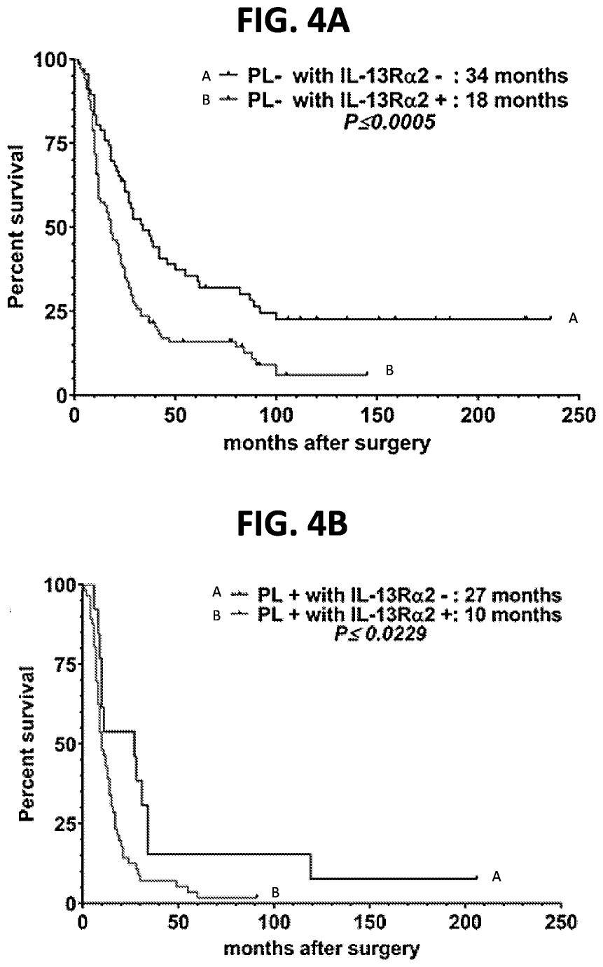

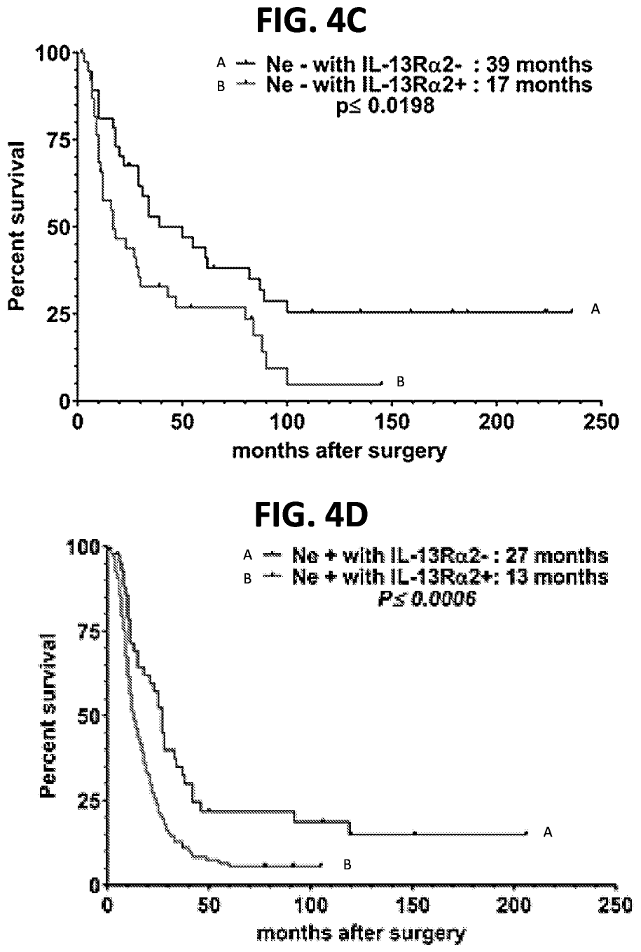

To evaluate the usefulness of IL-13Rα2 as an index of monitoring therapy and the natural history of PDAC patients, the results were further stratified and sub-categorized into four groups, each with PL− with IL-13Rα2−, PL− with IL-13Rα2+(FIG. 4A), PL+ with IL-13Rα2− and PL+ with IL-13Rα2+ (FIG. 4B) and studied their MST. MST for PL− plus IL-13Rα2− patients is significantly higher than that of PL− with IL-13Rα2+(34 months versus 18 months, P<0.0005). As shown in FIG. 4B, PL+ with IL-13Rα2− patients lived 17 months longer when compared to 10 months for PL+ with IL-13Rα2+ patients (P≤0.0229). Similarly, a significant increase in survival time was observed in patients with Ne− with IL-13Rα2− versus ne− with IL-13Rα2+ (FIG. 4C, P≤0.0198) and Ne+ with IL-13Rα2− versus ne+ with IL-13Rα2+ patients (FIG. 4D, P≤0.0006).

### Example 9

### Production and Purification of IL-13-BAD

A nucleic acid sequence encoding the IL-13-BAD immunotoxin was created and cloned into pET24a as described above in Example 1 (materials and methods, see also, FIG. 10). The IL-13-BAD plasmid was transformed into BL23(DE3)pLysS (E. coli) for protein expression. Expression of IL-13-BAD was induced by IPTG for 6 hours. The protein product was isolated and refolded in vitro. The protein was further purified by dialysis and ion-exchange chromatography. The resulting purified protein was used to test for biological activity of IL-13-BAD in vitro and in vivo.

### Example 10

### Cytotoxicity of IL-13-BAD

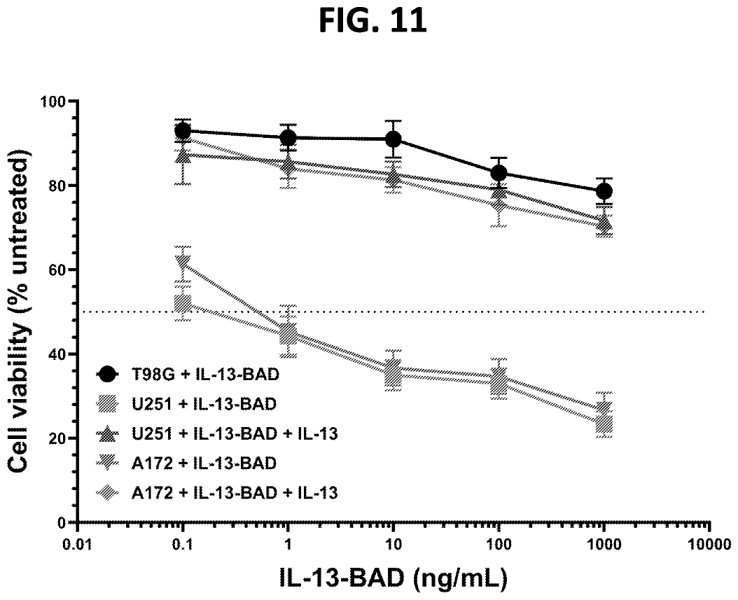

IL-13Rα2 positive (U251 and A172) and negative glioma (T98G) cells were cultured in the presence of different concentrations of IL-13-BAD for 5 days. IL-13-BAD showed concentration dependent specific killing of IL-13Rα2 positive cells lines U251 and A172 (FIG. 11). In contrast, cells that were incubated with 100X IL-13 for 2 hours prior to the addition of IL-13-BAD (IL-13-BAD+IL-13), were protected from the cytotoxic effect due to IL-13 blocking binding of the immunotoxin.

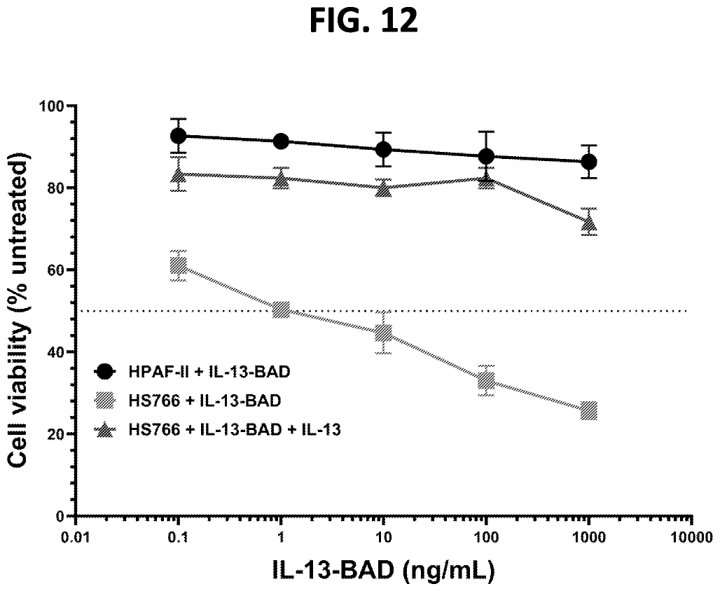

Similarly, IL-13Rα2 positive (HS766) and negative (HPAF-II) pancreatic tumor cells were cultured in presence of different concentrations of IL-13-BAD for 5 days. The results show IL-13-BAD specifically kills the IL-13Rα2 positive cells line (HS766+IL-13-BAD) (FIG. 12). In contrast, cells that were incubated with 100X IL-13 for 2 hours prior to the addition of IL-13-BAD (IL-13-BAD+IL-13), were protected from the cytotoxic effect.

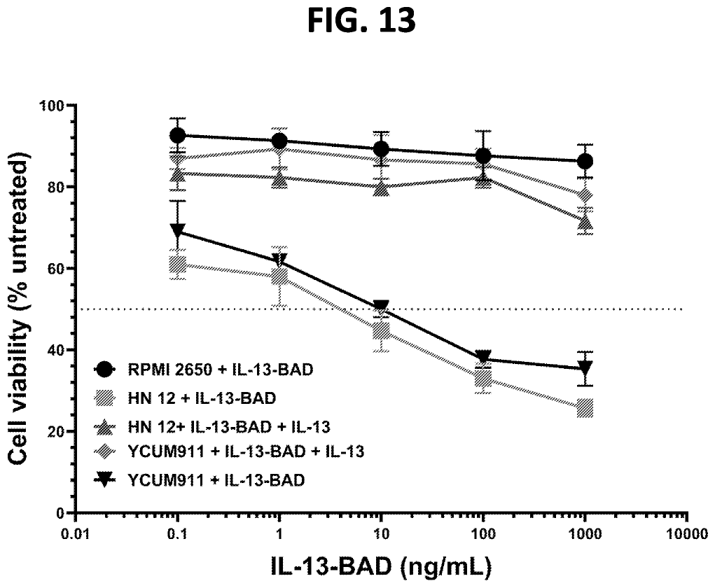

IL-13Rα2 positive (HN 12 and YCUM911) and negative (RPMI 2650) SCCHN cells were also cultured in presence of different concentrations of IL-13-BAD for five days. The results show that IL-13-BAD specifically kills IL-13Rα2 positive cells lines (HN 12 and YCUM911) (FIG. 13). In contrast, cells that were incubated with 100X IL-13 for 2 hours prior to the addition of IL-13-BAD (IL-13-BAD+IL-13), were protected from the cytotoxic effect.

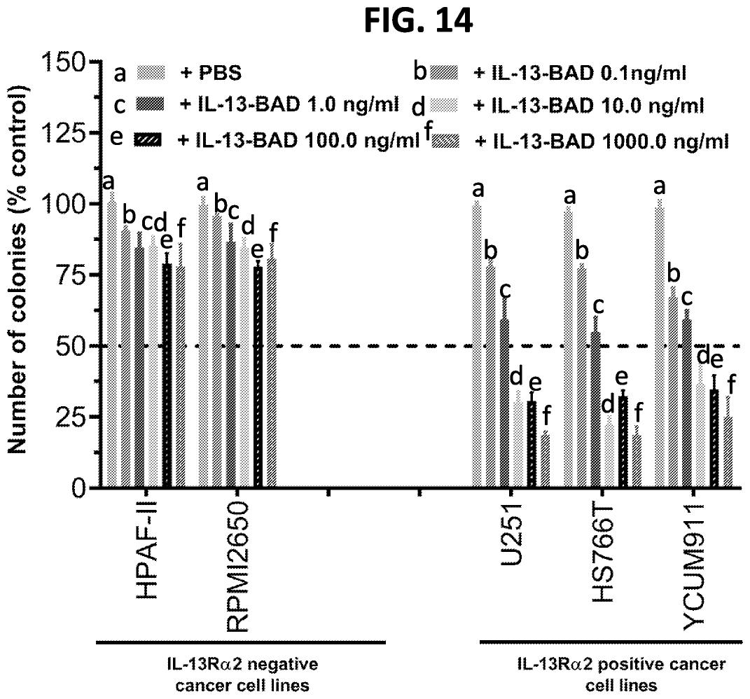

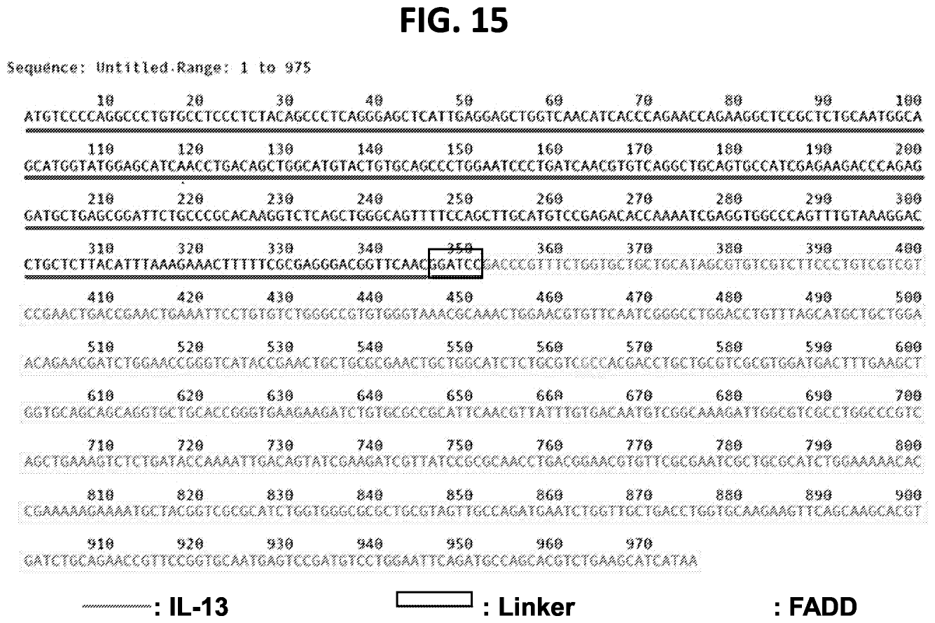

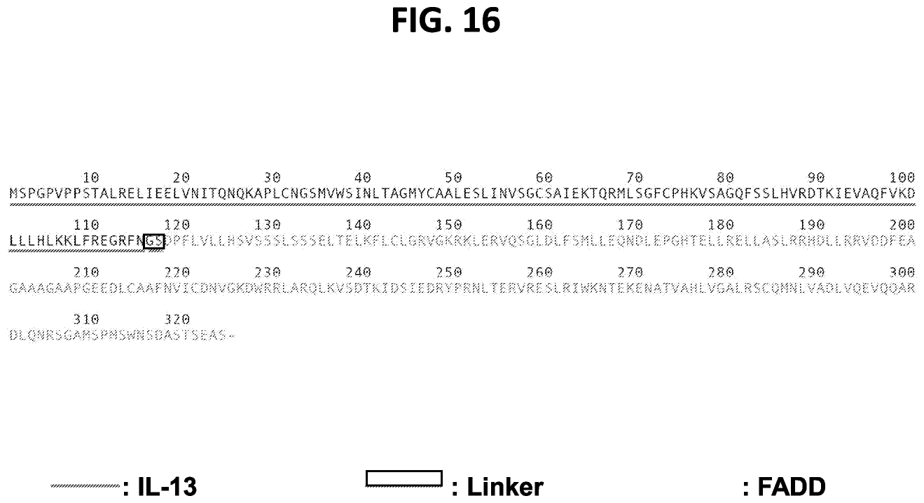

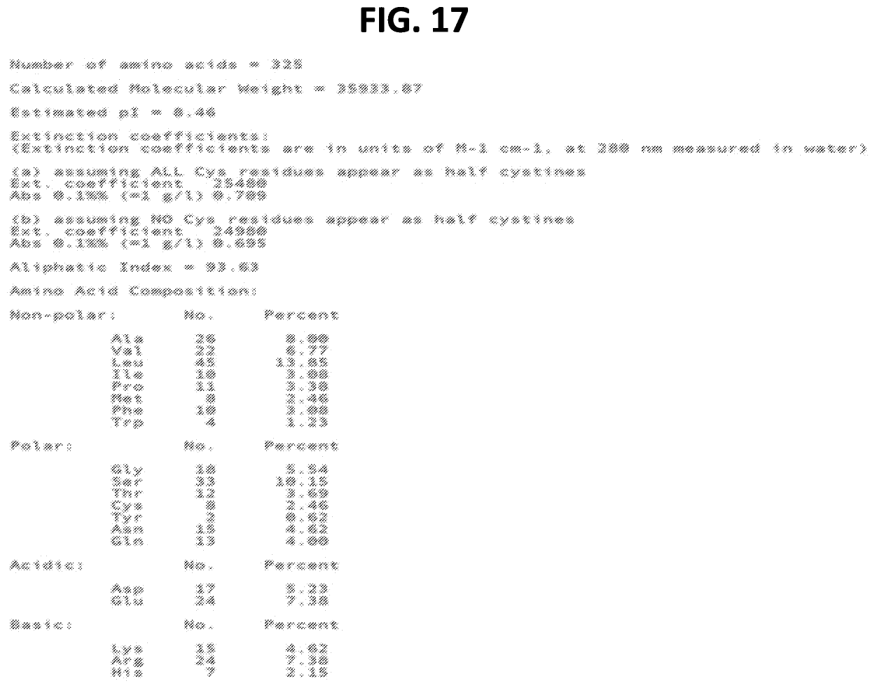

The colony forming ability of IL-13Rα2 positive and negative human tumor cells (U251, HS766T, and YCUM911) was also evaluated in the presence of different concentrations of IL-13-BAD (FIG. 14). The cells were incubated for a period of 8 days in a CO₂ incubator and then the number of colonies were counted (a colony formed with at least 50 or more tumor cells). A concentration dependent decline in number of colonies was observed in colonies formed by IL-13Rα2 positive tumor cells. In contrast, little effect was observed with the IL-13Rα2 negative tumor cells (HPAF-II and RPMI 2650).

### Example 11

### Safety and Efficacy of Immunotoxins In Vivo

This example describes methods that can be used to test safety and efficacy of IL-13Rα2 targeted immunotoxin (IL-13-PE, IL-13-BAD or IL-13-FADD) treatment in vivo. While particular methods are provided, one of skill in the art will recognize that methods that deviate from these specific methods can also be used, including addition or omission of one or more steps.

In this example, athymic nude immunodeficient mice are implanted subcutaneously or orthotopically with various human tumor cells derived from human brain tumor, pancreatic tumor (e.g., Panc-1 and ASPC-1), or other solid tumors. About 4-6 weeks after tumor implantation, the mice are treated with 50-100 μg/kg IL-13-immunotoxin (IL-13-PE, IL-13-BAD or IL-13-FADD) or placebo (PBS/0.2% human serum albumin-vehicle). The mice are examined for antitumor activity and immunological changes in response to treatment. For example, mice body weight and tumor size is measured every 4-7 days starting on day 4 after treatment with the immunotoxin. Immune cells such as CD4+, CD8+, T reg cell numbers, myeloid cells, cytokine levels are assessed in the blood at various time intervals. Tumor measurements continue until tumor size reaches 20 mm in diameter. An additional group of control animals are implanted with IL-13Rα2 knocked-down tumor cells and treated with these immunotoxins as described above.

To further enhance the efficacy of IL-13-PE, IL-13-BAD, or IL-13-FADD against solid tumors, about 4-6 weeks after tumor implantation (as described above), tumor bearing mice are pre-treated with 5-10 mg/kg TSA subcutaneously (s.c.) every alternative days or 25-50 mg/kg SAHA intraperitoneally (i.p.) daily for 14 days. The mice then receive 50-100 μg/kg IL-13-immunotoxin or placebo. The mice are examined for antitumor activity and immunological changes in response to treatment, as described above.

### Example 12

Clinical Trial Design for Treating PNI with IL-13 Immunotoxins

This example describes methods that can be used to test safety and efficacy of immunotoxins in a Phase I/II clinical trial in humans. While particular methods are provided, one of skill in the art will recognize that methods that deviate from these specific methods can also be used, including addition or omission of one or more steps.

Patients with pathological confirmation of PDAC and PNI lesions, and have a tumor positive (≥30%) for IL13Rα2 protein expression, are eligible for a Phase I/II clinical study. Patients receive prior or concurrent gemcitabine therapy. The study determines the maximal tolerated dose of IL-13-PE, IL-13-BAD, or IL-13-FADD and preliminary response to IL-13-PE, IL-13-BAD, or IL-13-FADD with a focus on PDAC and PNI-associated pain.

IL-13-PE, IL-13-BAD, or IL-13-FADD is administered periodically over a treatment course of 4 weeks, for example, intravenously on week 1, days 1, 3, and 5, of a 4-week cycle. Patients may receive up to 4 courses of treatment (16 weeks). Imaging studies and pain assessment start after the patient has completed one course of treatment and continue until the patient completes the treatment.

In view of the many possible embodiments to which the principles of the disclosure may be applied, it should be recognized that illustrated embodiments are only examples and should not be taken as limiting the scope of the invention. Rather, the scope of the invention is defined by the following claims. We therefore claim as our invention all that comes within the scope and spirit of these claims.

1. An immunotoxin, comprising:
a ligand, an antibody, or antibody fragment that specifically binds IL-13Rα2, linked to a mammalian cytotoxic protein.

2. The immunotoxin of claim 1, wherein the cytotoxic protein is a human protein.

3. (canceled)

4. The immunotoxin of claim 1, wherein the cytotoxic protein is BCL2 associated agonist of cell death (BAD) or Fas-associated death domain (FADD).

5. (canceled)

6. The immunotoxin of claim 1, wherein the ligand that specifically binds IL-13Rα2 is IL-13, a circularly permuted IL-13 (cpIL-13), or a fragment thereof.

7. The immunotoxin of claim 1, wherein the immunotoxin has at least 90% sequence identity to, or comprises or consists of, SEQ ID NO: 1 or SEQ ID NO: 3.

8. The immunotoxin of claim 1, wherein the antibody or antibody fragment comprises variable heavy chain (VH) domain complementarity determining region 1 (CDR1), CDR2, and CDR3 amino acid sequences of amino acid positions 31-35, 50-66, and 99-107 of SEQ ID NO: 11, respectively, and variable light chain (VL) domain complementarity determining region 1 (CDR1), CDR2, and CDR3 amino acid sequences of amino acid positions 157-167, 183-189, and 222-229 of SEQ ID NO: 11, respectively.

9. The immunotoxin of claim 8, wherein
(i) the antibody or antibody fragment comprises the variable heavy chain (VH) and/or variable light chain (VL) of SEQ ID NO: 11; and/or
(ii) the antibody or antibody fragment has at least 90% sequence identity to, or comprises or consists of, SEQ ID NO: 11.

10. (canceled)

11. A nucleic acid molecule encoding the immunotoxin of claim 1.

12. The nucleic acid molecule of claim 11, wherein the nucleic acid is codon-optimized for expression in a prokaryote.

13. The nucleic acid molecule of claim 11, comprising the ligand, wherein:
(i) the nucleic acid encodes the amino acid sequence of SEQ ID NO: 1 or SEQ ID NO: 3; and/or
(ii) the nucleic acid has at least 90% sequence identity to, or comprises or consists of, SEQ ID NO: 2 or SEQ ID NO: 4.

14. (canceled)

15. A vector comprising the nucleic acid molecule of claim 11.

16. The vector of claim 15, wherein the vector is a plasmid or viral vector.

17. A host cell expressing the immunotoxin of claim 1.

18. A host cell comprising the nucleic acid molecule of claim 11 or the or a vector comprising the nucleic acid molecule.

19. A method of producing the immunotoxin of claim 1, comprising transducing a host cell with a vector comprising a nucleic acid encoding the immunotoxin and expressing the nucleic acid molecule in the host cell, thereby producing the immunotoxin.

20. The method of claim 19, wherein the host cell is a bacterium, yeast, insect, or mammalian cell.

21. (canceled)

22. A method of treating an IL-13Rα2 expressing tumor or cancer in a subject, comprising administering an effective amount of the immunotoxin of claim 1 to the subject, thereby treating the IL-13Rα2 expressing tumor or cancer.

23. The method of claim 22, wherein the method reduces perineural invasion (PNI) of the IL-13Rα2 expressing tumor or cancer and/or reduces pain resulting from PNI.

24. (canceled)

25. The method of claim 23, further comprising selecting the subject having PNI for treatment.

26. A method of reducing perineural invasion (PNI) or reducing pain resulting from PNI of an IL-13Rα2 expressing cancer in a subject, comprising administering an effective amount of IL-13-PE immunotoxin to the subject, thereby reducing PNI or reducing pain from PNI.

27-28. (canceled)

29. The method of claim 22, wherein the subject has pancreatic cancer, head and neck cancer, ovarian cancer, cervical cancer, prostate cancer, stomach cancer, colorectal cancer, malignant glioma, Kaposi's sarcoma, kidney cancer, adrenocortical cancer, squamous cell carcinoma of skin, biliary tract tumor, vulvar carcinoma, oral cancer, or cholangiocarcinoma.

30. The method of claim 22, further comprising treating the subject with one or more of surgery, radiation, chemotherapy, biologic therapy, or immunotherapy.

31. The method of claim 22, further comprising administering to the subject a histone deacetylase (HDAC) inhibitor, checkpoint inhibitor, cell cycle or spindle assembly checkpoint inhibitor, adrenomedullin, or any combination of two or more thereof.


SEQUENCE LISTING (23 sequences)

SEQ ID NO: 1 - protein, 284 aa, Artificial Sequence
  Exemplary IL-13-BAD immunotoxin
MSPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGSFQIPEFEPSEQEDSSSAERGLGPSPAGDGPSGSGKHHRQAPGLLWDASHQQEQPTSSSHHGGAGAVEIRSRHSSYPAGTEDDEGMGEEPSPFRGRSRSAPPNLWAAQRYGRELRRMSDEFVDSFKKGLPRPKSAGTATQMRQSSSWTRVFQSWWDRNLGRGSSAPSQ

SEQ ID NO: 2 - DNA, 855 bp, Artificial Sequence
  Exemplary nucleic acid sequence encoding IL-13-BAD
atgtccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacggttcaacggatccttccagatcccagagtttgagccgagtgagcaggaagactccagctctgcagagaggggcctgggccccagccccgcaggggacgggccctcaggctccggcaagcatcatcgccaggccccaggcctcctgtgggacgccagtcaccagcaggagcagccaaccagcagcagccatcatggaggcgctggggctgtggagatccggagtcgccacagctcctaccccgcggggacggaggacgacgaagggatgggggaggagcccagcccctttcggggccgctcgcgctcggcgccccccaacctctgggcagcacagcgctatggccgcgagctccggaggatgagtgacgagtttgtggactcctttaagaagggacttcctcgcccgaagagcgcgggcacagcaacgcagatgcggcaaagctccagctggacgcgagtcttccagtcctggtgggatcggaacttgggcaggggaagctccgccccctcccagtga

SEQ ID NO: 3 - protein, 324 aa, Artificial Sequence
  Exemplary IL-13-FADD immunotoxin
MSPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNGSDPFLVLLHSVSSSLSSSELTELKFLCLGRVGKRKLERVQSGLDLFSMLLEQNDLEPGHTELLRELLASLRRHDLLRRVDDFEAGAAAGAAPGEEDLCAAFNVICDNVGKDWRRLARQLKVSDTKIDSIEDRYPRNLTERVRESLRIWKNTEKENATVAHLVGALRSCQMNLVADLVQEVQQARDLQNRSGAMSPMSWNSDASTSEAS

SEQ ID NO: 4 - DNA, 975 bp, Artificial Sequence
  Exemplary nucleic acid sequence encoding IL-13-FADD
atgtccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacggttcaacggatccgacccgtttctggtgctgctgcatagcgtgtcgtcttccctgtcgtcgtccgaactgaccgaactgaaattcctgtgtctgggccgtgtgggtaaacgcaaactggaacgtgttcaatcgggcctggacctgtttagcatgctgctggaacagaacgatctggaaccgggtcataccgaactgctgcgcgaactgctggcatctctgcgtcgccacgacctgctgcgtcgcgtggatgactttgaagctggtgcagcagcaggtgctgcaccgggtgaagaagatctgtgcgccgcattcaacgttatttgtgacaatgtcggcaaagattggcgtcgcctggcccgtcagctgaaagtctctgataccaaaattgacagtatcgaagatcgttatccgcgcaacctgacggaacgtgttcgcgaatcgctgcgcatctggaaaaacaccgaaaaagaaaatgctacggtcgcgcatctggtgggcgcgctgcgtagttgccagatgaatctggttgctgacctggtgcaagaagttcagcaagcacgtgatctgcagaaccgttccggtgcaatgagtccgatgtcctggaattcagatgccagcacgtctgaagcatcataa

SEQ ID NO: 5 - protein, 167 aa, Artificial Sequence
  Exemplary cytotoxic BAD protein
FQIPEFEPSEQEDSSSAERGLGPSPAGDGPSGSGKHHRQAPGLLWDASHQQEQPTSSSHHGGAGAVEIRSRHSSYPAGTEDDEGMGEEPSPFRGRSRSAPPNLWAAQRYGRELRRMSDEFVDSFKKGLPRPKSAGTATQMRQSSSWTRVFQSWWDRNLGRGSSAPSQ

SEQ ID NO: 6 - DNA, 504 bp, Artificial Sequence
  Exemplary nucleic acid sequence encoding a cytotoxic BAD protein
ttccagatcccagagtttgagccgagtgagcaggaagactccagctctgcagagaggggcctgggccccagccccgcaggggacgggccctcaggctccggcaagcatcatcgccaggccccaggcctcctgtgggacgccagtcaccagcaggagcagccaaccagcagcagccatcatggaggcgctggggctgtggagatccggagtcgccacagctcctaccccgcggggacggaggacgacgaagggatgggggaggagcccagcccctttcggggccgctcgcgctcggcgccccccaacctctgggcagcacagcgctatggccgcgagctccggaggatgagtgacgagtttgtggactcctttaagaagggacttcctcgcccgaagagcgcgggcacagcaacgcagatgcggcaaagctccagctggacgcgagtcttccagtcctggtgggatcggaacttgggcaggggaagctccgccccctcccagtga

SEQ ID NO: 7 - protein, 207 aa, Artificial Sequence
  Exemplary cytotoxic FADD protein
DPFLVLLHSVSSSLSSSELTELKFLCLGRVGKRKLERVQSGLDLFSMLLEQNDLEPGHTELLRELLASLRRHDLLRRVDDFEAGAAAGAAPGEEDLCAAFNVICDNVGKDWRRLARQLKVSDTKIDSIEDRYPRNLTERVRESLRIWKNTEKENATVAHLVGALRSCQMNLVADLVQEVQQARDLQNRSGAMSPMSWNSDASTSEAS

SEQ ID NO: 8 - DNA, 630 bp, Artificial Sequence
  Exemplary nucleic acid sequence encoding a cytotoxic FADD protein
gacccgtttctggtgctgctgcatagcgtgtcgtcttccctgtcgtcgtccgaactgaccgaactgaaattcctgtgtctgggccgtgtgggtaaacgcaaactggaacgtgttcaatcgggcctggacctgtttagcatgctgctggaacagaacgatctggaaccgggtcataccgaactgctgcgcgaactgctggcatctctgcgtcgccacgacctgctgcgtcgcgtggatgactttgaagctggtgcagcagcaggtgctgcaccgggtgaagaagatctgtgcgccgcattcaacgttatttgtgacaatgtcggcaaagattggcgtcgcctggcccgtcagctgaaagtctctgataccaaaattgacagtatcgaagatcgttatccgcgcaacctgacggaacgtgttcgcgaatcgctgcgcatctggaaaaacaccgaaaaagaaaatgctacggtcgcgcatctggtgggcgcgctgcgtagttgccagatgaatctggttgctgacctggtgcaagaagttcagcaagcacgtgatctgcagaaccgttccggtgcaatgagtccgatgtcctggaattcagatgccagcacgtctgaagcatcataaaagctt

SEQ ID NO: 9 - protein, 115 aa, Artificial Sequence
  Exemplary matured human IL-13 protein
MSPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 10 - DNA, 345 bp, Artificial Sequence
  Exemplary nucleic acid sequence encoding matured human IL-13
atgtccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacggttcaac

SEQ ID NO: 11 - protein, 241 aa, Artificial Sequence
  Exemplary IL-13Ra2 scFv
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARMNHMIPLKAWGQGTLVTVSSGGGGSGGGGSGGSALAIQMTQSPSSLSASVGDRVTITCRASQGIRNDLGWYQQKPGKAPKLLIYAASSLQSGVPSRFSGSGSGTDFTLTISSLQPEDFATYYCLQMYNYRTFGQGTKLEIKRA

SEQ ID NO: 12 - DNA, 722 bp, Artificial Sequence
  Exemplary nucleic acid sequence encoding the IL-13Ra2 scFv
aggtgcagctggtgcagagcggagcagaggtgaagaagccaggagcctctgtgaaggtgagctgcaaggcctccggctacacattcacctcctatgccatgcactgggtgagacaggcacctggacagaggctggagtggatgggctggatcaacgccggcaacggcaatacaaagtactctcagaagtttcagggccgcgtgacaatcacccgggacacatccgcctctaccgcctatatggagctgagctccctgcggtccgaggataccgccgtgtactattgcgccagaatgaatcacatgatcccactgaaggcatggggacagggcacactggtgaccgtgtctagcggaggaggaggcagcggaggaggaggctccggcggctctgccctggccatccagatgacccagtccccatcctctctgagcgcctccgtgggcgaccgcgtgacaatcacctgtcgggccagccagggcatcagaaacgatctgggctggtaccagcagaagcccggcaaggcccctaagctgctgatctatgcagcaagctccctgcagtctggagtgcctagccggttctctggcagcggctccggaacagactttacactgaccatctctagcctgcagccagaggatttcgccacctactattgcctgcagatgtacaattatagaacatttggccagggcaccaagctggagatcaagagggcc

SEQ ID NO: 13 - DNA, 27 bp, Artificial Sequence
  Exemplary primer sequence
taatttgcccatatgtccccaggccct

SEQ ID NO: 14 - DNA, 30 bp, Artificial Sequence
  Exemplary primer sequence
gaagttggatcctgttgaaccgtccctcgc

SEQ ID NO: 15 - DNA, 27 bp, Artificial Sequence
  Exemplary primer sequence
gttaaaggatccgacccgtttctgctc

SEQ ID NO: 16 - DNA, 24 bp, Artificial Sequence
  Exemplary primer sequence
tcctgtaagctttcaggacgcttc

SEQ ID NO: 17 - protein, 467 aa, Artificial Sequence
  Exemplary IL-13-PE immunotoxin
MSPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNKASGGPEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDQEQAISALPDYASQPGQPPREDLR

SEQ ID NO: 18 - DNA, 1502 bp, Artificial Sequence
  Exemplary nucleic acid sequence encoding an IL-13-PE immunotoxin
atgtccccaggccctgtgcctccctctacagccctcagggagctcattgaggagctggtcaacatcacccagaaccagaaggctccgctctgcaatggcagcatggtatggagcatcaacctgacagctggcatgtactgtgcagccctggaatccctgatcaacgtgtcaggctgcagtgccatcgagaagacccagaggatgctgagcggattctgcccgcacaaggtctcagctgggcagttttccagcttgcatgtccgagacaccaaaatcgaggtggcccagtttgtaaaggacctgctcttacatttaaagaaactttttcgcgagggacggttcaacaaagcttccggaggtcccgagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatcccggaccaagaacaggcgatcagcgccctgccggactacgccagccagcccggccaaccgccgcgcgaggacctgcgttaactgccgcgaccggccggctcccttcgcaggagccggccttctcggggcctggccatacatcaggttttcctgatgccagcccaatcgaatatgaattc

SEQ ID NO: 19 - protein, 168 aa, Homo sapiens
MFQIPEFEPSEQEDSSSAERGLGPSPAGDGPSGSGKHHRQAPGLLWDASHQQEQPTSSSHHGGAGAVEIRSRHSSYPAGTEDDEGMGEEPSPFRGRSRSAPPNLWAAQRYGRELRRMSDEFVDSFKKGLPRPKSAGTATQMRQSSSWTRVFQSWWDRNLGRGSSAPSQ

SEQ ID NO: 20 - protein, 208 aa, Homo sapiens
MDPFLVLLHSVSSSLSSSELTELKFLCLGRVGKRKLERVQSGLDLFSMLLEQNDLEPGHTELLRELLASLRRHDLLRRVDDFEAGAAAGAAPGEEDLCAAFNVICDNVGKDWRRLARQLKVSDTKIDSIEDRYPRNLTERVRESLRIWKNTEKENATVAHLVGALRSCQMNLVADLVQEVQQARDLQNRSGAMSPMSWNSDASTSEAS

SEQ ID NO: 21 - protein, 15 aa, Artificial Sequence
  Exemplary amino acid linker
GGGGSGGGGSGGGGS

SEQ ID NO: 22 - protein, 12 aa, Artificial Sequence
  Exemplary amino acid linker
TRHRQPRGWEQL

SEQ ID NO: 23 - protein, 132 aa, Homo sapiens
MALLLTTVIALTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN